(19) United States
(12) Patent Application Publication
(10) Pub. No.: US 2026/0041095 A1
(43) Pub. Date: Feb. 12, 2026

(54) A PRESERVATION METHOD

(71) Applicant: AnalytiCon Discovery - Zweigniederlassung der BRAIN Biotech AG, Potsdam (DE)

(72) Inventors: Werner KATZER, Berlin (DE); Karsten SIEMS, Michendorf (DE); Axel THELITZ, Berlin (DE); Yvonne TIFFERT, Mannheim (DE)

(21) Appl. No.: 19/099,513

(22) PCT Filed: Jul. 31, 2023
(86) PCT No.: PCT/EP2023/071152

§ 371 Date: Jan. 29, 2025

(30) Foreign Application Priority Data
Aug. 5, 2022 (EP) 22188936.3

(51) Int. Cl. A01N 47/06; A01P 1/00; A01P 3/00; A61K 8/60; C11D 3/48

(52) U.S. Cl. CPC A01N 47/06; A01P 1/00; A01P 3/00; A61K 8/602; C11D 3/48

(57) ABSTRACT

Suggested is a method for improving antimicrobial stability of a composition comprising or consisting of glycolipids, said glycolipids being selected from at least one ustilagic acid according to formula (I)-(III) or compounds 1-6.

### AREA OF INVENTION

The present invention refers to the area of glycolipids and concerns new and effective methods for preservation of preparations, by using anti-microbial agents of natural origin.

### BACKGROUND OF THE INVENTION

The rise of resistant microbes illustrates the need for further compounds able to prevent microbial infections and contaminations, while showing low toxicity in humans. Some glycolipids such as ustilagic acid have demonstrated anti-microbial properties. For instance, glycolipids have been shown to interact with the lipid bilayer of microorganisms thereby leading to deformation and collapse of the membrane skeleton. Glycolipid treatment is also associated with increased generation of reactive oxygen species (ROS) in bacteria and fungi. Accumulation of ROS occurs due to insufficient cellular detoxification. It is commonly observed in microorganisms following treatment with membrane disruptors and antibiotics and can lead to cellular apoptosis. Therefore, glycolipids show great potential for anti-microbial applications due to their high biological activity against microbes with low observed toxicity.

Glycolipids are particularly useful as food preservatives but also for other products of daily life, e.g. pet food and cosmetics. Preservation methods such as pasteurization or addition of chemical preservatives such as sorbic or benzoic acid can have a profound impact on the sensory properties of the product. However, sensory changes of products upon preservation are undesirable as customers value natural flavor. Additionally, there is a need for alternatives to synthetically produced conservatives due to the high environmental cost of precursor compounds used for industrial production. Glycolipids represent a suitable alternative to widely used preservation approaches due to their anti-microbial properties, low toxicity and more eco-friendly production.

### RELEVANT PRIOR ART

Glycolipids including cellobiose glycolipids (e.g. ustilagic acids) are well known as surfactants and showed moderate antimicrobial activity due to the disruption of the cell wall of the microorganisms, especially of yeasts and gram positive bacteria [Qin Shu et al. Pharmaceutics 2021, 13, 227, B. Mimee et al. Journal of Applied Microbiology 107 (2009) 989-996].

WO 2013 037818 A1 (WACKER) discloses ustilagic acid and its esters for fighting specific germs such as Alicyclobacillus acidoterrestris, Penicillium expansum, Gluconacetobacter liquefaciens, Lactiplantibacillus plantarum, Zygosaccharomyces rouxii, Aspergillus brasiliensis, Brettanomyces naardanensis, and Weissella confusa in non-alcoholic beverages.

### OBJECT OF THE INVENTION

Due to increasing resistance of specific microbes, particularly of germs like
Asaia siamensis
Bacillus licheniformis
Bacillus subtilis
Byssochlamys fulva
Byssochlamys nivea
Candida parapsilosis
Clostridium perfringens
Clostridium sporogenes
Corynebacterium xerosis
Enterococcus faecalis
Lactobacillus paracasei ssp. paracasei
Leuconostoc lactis
Leuconostoc mesenteroides
Malassezia furfur
Neosartorya fischeri
Penicillium paneum
Saccharomyces cerevisiae
Yarrowia lipolytica
Zygosaccharomyces rouxii
which are found in many products of daily life, there is demand for new and highly effective preservatives, particularly obtained from natural sources and capable of fighting these microbes, preferably at a very low application level.

The preservative composition of the present invention is preferably applied for food products excluding beverages.

### BRIEF DESCRIPTION OF THE INVENTION

A first object of the present invention refers to a method for improving antimicrobial stability of a composition, such as cosmetic, detergent or food preparations, against at least one of the following micro-organisms
Asaia siamensis
Bacillus licheniformis
Bacillus subtilis
Byssochlamys fulva
Byssochlamys nivea
Candida parapsilosis
Clostridium perfringens
Clostridium sporogenes
Corynebacterium xerosis
Enterococcus faecalis
Lactobacillus paracasei ssp. paracasei
Leuconostoc lactis
Leuconostoc mesenteroides
Malassezia furfur
Neosartorya fischeri
Penicillium paneum
Saccharomyces cerevisiae
Yarrowia lipolytica
Zygosaccharomyces bailii
   comprising or consisting of the following steps:
(a) providing a composition in need of such improved antimicrobial stability;
(b) providing at least one glycolipid and optionally at least one other antimicrobial agent;
(c) adding a working amount of said at least one glycolipid and optionally said at least one other anti-microbial agent to said composition;
(d) optionally providing at least one other compound to said composition affecting solubility of compounds added in step (b) and (c);
(e),
   wherein said glycolipid represents an ustilagic acid or an ester of ustilagic acid esterified with a linear C₁-C₄ aliphatic alcohol. In some embodiments said method further comprises step (e) optionally adjusting the pH of the composition thus obtained to about 1 to about 4.

### Glycolipids

Glycolipids are molecules with at least one glycosidic bond between mono- or oligosaccharides and lipid molecules. Several classes of glycolipids have been identified such as rhamnolipids, trehaloselipids, cellobioselipids and mannosylerythritolipids.

Ustilagic acid is an organic compound with the formula C₃₆H₆₄O₁₈. The acid is a cellobiose lipid produced by the corn smut fungus Ustilago maydis under conditions of nitrogen starvation. The acid was discovered in 1950 and was proven to be an amphipathic glycolipid with surface active properties. The name comes from Latin ustus which means burnt and refers to the scorched appearance of the smut fungi. Yeast species of the genus Pseudozyma are known to produce ustilagic acid. For example, the yeasts Pseudozyma fusiformata and Pseudozyma graminicola secrete ustilagic acids, 2-O-3-hydroxyhexanoyl-beta-D-glucopyranosyl-(1→4)-6-O-acetyl-beta-D-glucopyranosyl-(1→16)-2,15,16-trihydroxyhexadecanoic acid. Similar compounds are the extracellular cellobiose lipids of the yeasts Cryptococcus humicola and Trichosporon porosum: 2,3,4-O-triacetyl-beta-D-glucopyranosyl-(1→4)-6-O-acetyl-beta-D-glucopyranosyl-(1→16)-2,16-dihydroxyhexa decanoic acid.

Some glycolipids can be used as natural conservatives without changing the sensatory properties of the product. Ustilagic acids have been shown to possess antibiotic properties (U.S. Pat. No. 2,698,843). The biosynthesis of ustilagic acid as well as its use as compound controlling the phytopathogen Botrytis cinerea have been reported before (http://archiv.ub.uni-marburg .de/opus/frontdoor.php?source gpus=234 2&la=de).

Surprisingly it has been found that the glycolipids according to the present invention are active against a broad spectrum of microorganisms, including bacteria, fungi and yeasts, particularly
Asaia siamensis
Bacillus licheniformis
Bacillus subtilis
Byssochlamys fulva
Byssochlamys nivea
Candida parapsilosis
Clostridium perfringens
Clostridium sporogenes
Corynebacterium xerosis
Enterococcus faecalis
Lactobacillus paracasei ssp. paracasei
Leuconostoc lactis
Leuconostoc mesenteroides
Malassezia furfur
Neosartorya fischeri
Penicillium paneum
Saccharomyces cerevisiae
Yarrowia lipolytica
Zygosaccharomyces bailii
   already at very low working levels given that the respective compositions are adjusted to low pH values of from about 1 to about 4, preferably of from about 1 to about 3 and more preferably of about 2 to about 2.5 The products are typically obtained or obtainable by extraction of natural sources.

Glycolipids according to the present invention are known to possess excellent emulsifying and surfactant properties. Therefore, in a preferred embodiment, the glycolipids according to the present invention substitute or support emulsifiers and surfactant present in compositions according to the present invention.

### Preferred Glycolipids

In a preferred embodiment the glycolipids according to the present invention follow formula (I)

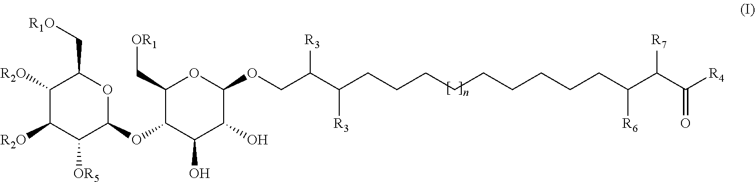

wherein independently from each other
n stands for 1 or 2,
R₁ represents hydrogen or —COCH₃;
R₂ represents H or

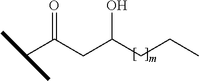

m=1, 2 or 3;
R₃ represents hydrogen or —OH;
R₄ represents —OH or —OCH₃;
R₅ represents hydrogen or

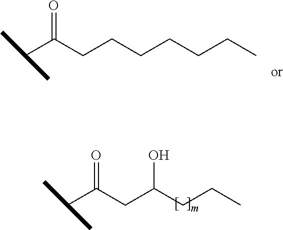

or m=1, 2 or 3;
R₆ represents hydrogen or —OH;
R₇ represents hydrogen or —OH or ═O;
or a salt of said glycolipid.

In a preferred embodiment, the glycolipids according to the present invention follow formula (II)

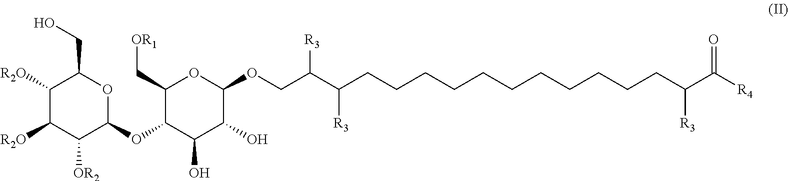

wherein independently from each other
R₃ represents hydrogen or —COCH₃;
R₂ represents H or

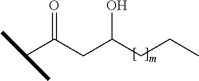

m=1, 2 or 3;
R₃ represents hydrogen or —OH;
R₄ represents —OH or —OCH3;
or a salt of said glycolipid.

In a preferred embodiment, the glycolipids according to the present invention follow formula (III)

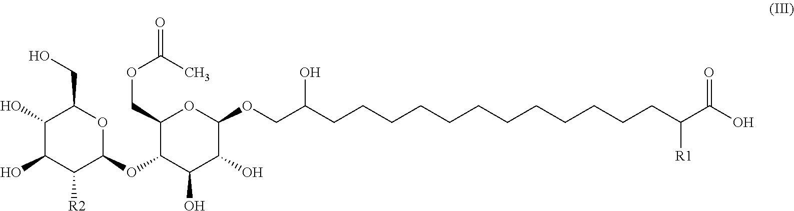

wherein independently from each other
R₁ represents hydrogen or —OH;
R₂ represents hydrogen or

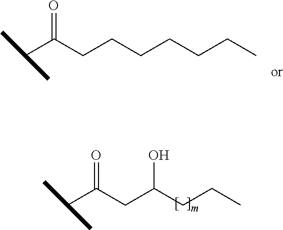

or m=1, 2 or 3;
or a salt of said glycolipid.

Surprisingly, while all antimicrobial compounds of the present invention are active against most of the microorganisms mentioned above, certain compounds show an exceptionally high and broad activity. In particular compounds following the general structure of formula III are able to inhibit growth and viability of microorganisms at even lower working concentrations compared to other glycolipids.

Compounds 1 to 6, following the general formula of formula (III), in particular exhibit excellent antimicrobial properties at low concentrations. In a preferred embodiment the glycolipids according to the present invention are selected from the following formulae of Compound 1 to 6:

| Compound | Structure |
|---|---|
| 1 | 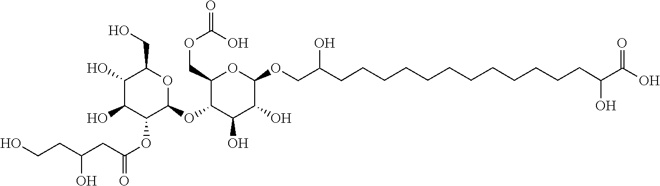 [SMILES: O=C(O)OC[C@H]1O[C@@H](OCC(O)CCCCCCCCCCCCC(O)C(=O)O)[C@H](O)[C@@H](O)[C@@H]1O[C@@H]1O[C@H](CO)[C@@H](O)[C@H](O)[C@H]1OC(=O)CC(O)CCO] |
| 2 | 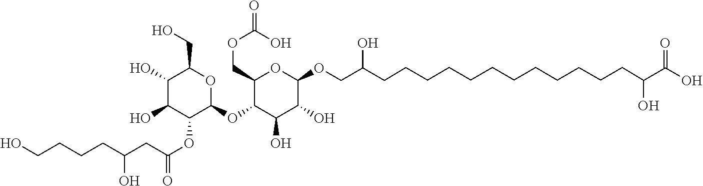 [SMILES: O=C(O)OC[C@H]1O[C@@H](OCC(O)CCCCCCCCCCCCC(O)C(=O)O)[C@H](O)[C@@H](O)[C@@H]1O[C@@H]1O[C@H](CO)[C@@H](O)[C@H](O)[C@H]1OC(=O)CC(O)CCCCO] |
| 3 | 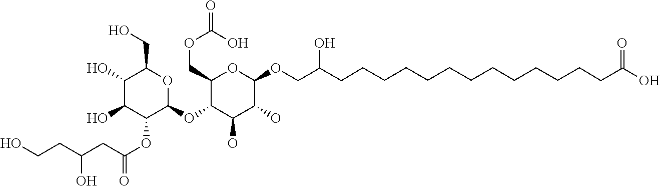 [SMILES: O=C(O)CCCCCCCCCCCCCC(O)CO[C@@H]1O[C@H](COC(=O)O)[C@@H](O[C@@H]2O[C@H](CO)[C@@H](O)[C@H](O)[C@H]2OC(=O)CC(O)CCO)[C@H](O)[C@H]1O] |
| 4 | 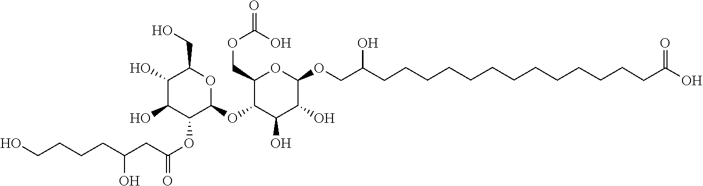 [SMILES: O=C(O)CCCCCCCCCCCCCC(O)CO[C@@H]1O[C@H](COC(=O)O)[C@@H](O[C@@H]2O[C@H](CO)[C@@H](O)[C@H](O)[C@H]2OC(=O)CC(O)CCCCO)[C@H](O)[C@H]1O] |
| 5 | 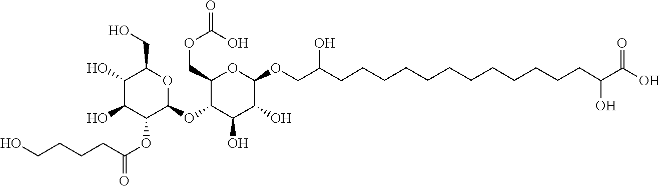 [SMILES: O=C(O)OC[C@H]1O[C@@H](OCC(O)CCCCCCCCCCCCC(O)C(=O)O)[C@H](O)[C@@H](O)[C@@H]1O[C@@H]1O[C@H](CO)[C@@H](O)[C@H](O)[C@H]1OC(=O)CCCCO] |
| 6 | 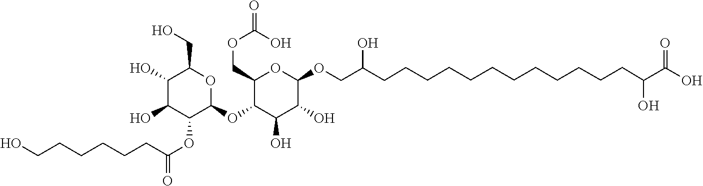 [SMILES: O=C(O)OC[C@H]1O[C@@H](OCC(O)CCCCCCCCCCCCC(O)C(=O)O)[C@H](O)[C@@H](O)[C@@H]1O[C@@H]1O[C@H](CO)[C@@H](O)[C@H](O)[C@H]1OC(=O)CCCCCCO] |

| | | Molecular | |
|---|---|---|---|
| Compound | Formula | weight | CAS |
| 1 | C₃₆H₆₄O₁₈ | 784.89 | 867578-35-6 |
| 2 | C₃₈H₆₈O₁₈ | 812.94 | 1427087-16-8 |
| 3 | C₃₆H₆₄O₁₇ | 768.89 | 1427028-86-1 |
| 4 | C₃₈H₆₈O₁₇ | 796.95 | |
| 5 | C₃₆H₆₄O₁₇ | 768.89 | |
| 6 | C₃₈H₆₈O₁₇ | 796.95 | 1427039-33-5 |

or a salt of said glycolipid.

In a preferred embodiment the salts according to the present invention represent alkali salts, alkaline salts, alkaline earth salts, ammonium salts, alkanol ammonium salts, glucammonium salts or mixtures thereof.

In a preferred embodiment at least one glycolipid according to the present invention is added in an amount of from 1 to 1,000 ppm—calculated on composition.

In a preferred embodiment at least one glycolipid according to the present invention is added in an amount of from 10 to 200 ppm—calculated on composition.

In a preferred embodiment of the method the pH value of the composition is adjusted by inorganic or organic salts.

### Secondary Additional Anti-Microbial Compounds

In a preferred embodiment the preservative method according to the present invention may include blends of said glycolipids, additional antimicrobial compounds different from said glycolipids as they are for example listed in REGULATION (EC) No 1333/2008 OF THE EUROPEAN PARLIAMENT AND OF THE COUNCIL of 16 Dec. 2008 on food additives or in REGULATION (EC) No 1223/2009 OF THE EUROPEAN PARLIAMENT AND OF THE COUNCIL of 30 Nov. 2009 on cosmetic products as well as further compounds affecting solubility of said glycolipids such as saponins or emulsifiers listed in EU 1333/2008 or EU 1223/2009.

Suitable species can be selected from the group consisting of preservatives selected from the group consisting of benzoic acid and para-hydroxybenzoic acid, their esters and salts, benzyl benzoate, propionic acid and its salts, salicylic acid and its salts, 2,4-hexadienoic acid (sorbic acid) and its salts, levulinic acid and its salts, anisic acid and its salts, perillic acid and its salts, cinnamic acid and its salts, formaldehyde and paraformaldehyde, 4-hydroxy benzaldehyde, ortho-, meta-, and para-anisic aldehyde, cinnamic aldehyde, cinnamic alcohol, 2-hydroxybiphenyl ether and its salts, 2-zinc-sulfidopyridine N-oxide, inorganic sulfites and bisulfites, sodium iodate, chlorobutanolum, 4-ethylmercury-(II)5-amino-1,3-bis(2-hydroxybenzoic acid), its salts and esters, dehydracetic acid, formic acid, 1,6-bis(4-amidino-2-bromophenoxy)-n-hexane and its salts, the sodium salt of ethylmercury-(II)-thiosalicylic acid, phenylmercury and its salts, 10-undecylenic acid and its salts, 5-amino-1,3-bis(2-ethylhexyl)-5-methyl-hexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)-urea, 4-chloro-m-cresol, 2,4,4′-trichloro-2′-hydroxy-diphenyl ether, 4-chloro-3,5-dimethylphenol, 1,1′-methylene-bis(3-(1-hydroxymethyl-2,4-dioximidazolidin-5-yl)urea), poly-(hexamethylenediguanide) hydrochloride, (Benzyloxymethoxy)-methanol hexamethylenetetramine, 1-(3-chloroallyl)-3,5,7-triaza-1-azonia-adamantane chloride, 1-(4-chlorophenoxy)-1-(1H-imidazol-1-yl)-3,3-dimethyl-2-butanone, 1,3-bis-(hydroxymethyl)-5,5-dimethyl-2,4-imidazolidinedione, 1,2-dibromo-2,4-dicyanobutane, 2,2′-methylene-bis(6-bromo-4-chlorophenol), bromochlorophene, mixture of 5-chloro-2-methyl-3(2H)-isothiazolinone, 2-methyl-3(2H)-isothiazolinone and with magnesium chloride and magnesium nitrate, 2-Octyl-2H-isothiazol-3-one, 1,2-benzisothiazol-3(2H)-one, 2-benzyl-4-chlorophenol, 3-(4-Chlorphenoxy)-1,2-propanediol (Chlorphenesin), 2-chloroacetamide, chlorhexidine, chlorhexidine acetate, chlorhexidine gluconate, chlorhexidine hydrochloride, N-alkyl(C₁₂-C₂₂)trimethyl-ammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxymethylurea, 1,6-bis(4-amidino-phenoxy)-n-hexane and its salts, glutaraldehyde, 5-ethyl-1-aza-3,7-dioxabicyclo(3.3.0)octane, 3-(4-chlorophenoxy)-1,2-propanediol, hyamines, alkyl-(C₈-C₁₈)dimethyl-benzyl-ammonium chloride, alkyl-(C₈-C₁₈)-dimethyl-benzylammonium bromide, alkyl-(C₈-Cₗ₈)-dimethyl-benzyl-ammonium saccharinate, benzyl hemiformal, 3-iodo-2-propynyl butylcarbamate, sodium hydroxymethyl-aminoacetate or sodium hydroxymethyl-aminoacetate, imidazolidinylurea, diazolidinylurea, sodium hydroxymethylglycinate, DMDM hydantoin, Tropolone, (Ethylendioxy)dimethanol, 2-Brom-2-(brommethyl)pentandinitril, N-(3-Aminopropyl)-N-dodecylpropan-1,3-diamin, α,α′,α″38-trimethyl-1,3,5-triazine-1,3,5(2H,4H,6H)-triethanol, pyridine-2-thiol-1-oxide, sodium salt, Tetrahydro-1,3,4,6-tetrakis(hydroxymethyl)imidazo[4,5-d]10erillart-2,5(1H,3H)-dion, 1,3-bis(hydroxymethyl)-1-(1,3,4-tris(hydroxy-methyl)-2,5-dioxoimidazolidin-4-yl)urea (Diazolidinyl Urea), 1,3-Bis(hydroxy-methyl)-5,5-dimethylimidazolidine-2,4-dione, 3-Acetyl-2-hydroxy-6-methyl-4H-pyran-4-one, cetyl pyridium chloride, caprylhydroxamic acid, sorbohydroxamic acid and their mixtures; 1,3-propanediol, methyl propanediol, 1,2-pentanediol, 1,2-hexanediol, 1,2-octanediol, 1,2-decanediol, 1,5-pentanediol, 1,6-hexanediol, 1,8-octanediol, 1,2-decanediol, ethylhexylglycerin, hexoxy-propan-1,2-diol, heptoxy-propan-1,2-diol, octoxy-propan-1,2-diol, 3-phenoxy-propan-1,2-diol, 3-benzyloxypropan-1,2-diol, 3-phenylethyloxy-propan-1,2-diol, 3-phenylpropyloxy-propan-1,2-diol, 3-methylbenzyloxy-propan-1,2-diol, sorbitan caprylate, triclosan, climbazole, Octopirox (1-hydroxy-4-methyl-6-(2,4,4-trimethylpentyl)-2(1H)-pyridone, 2-aminoethanol), chitosan, farnesol, 2-butyloctanoic acid, 2-Benzylheptan-1-ol, glycerol monolaurate, bis(2-pyridylthio)zinc 1,1′-dioxide, N,N′-(decane-1,10-diyldipyridin-1-yl-4-ylidene)-dioctan-1-amine dihydrochloride (octenidine dihydrochloride), thymol, eugenol, benzyl alcohol, 2-phenyethyl alcohol, 3-phenyl propanol, 2-phenoxyethanol, 1-phenoxy-propan-2-ol, 3-phenoxypropanol, benzyloxymethanol, 4-hydroxyacetophenone, lactic acid, ethanol, PHB (esters). and mixtures thereof.

In a preferred embodiment, secondary additional anti-microbial compounds are selected from the group of benzoic acid and 2,4-hexadienoic acid (sorbic acid), lactic acid, ethanol and para-hydroxybenzoic acid (PHB), their esters and salts.

### Solubilizers

A further objective of the present invention has been improving solubility of glycolipids and their mixtures in aqueous solutions depending on the selected glycolipids and other compounds as well as the desired pH value. It is therefore beneficial for the present invention to further add compounds that increase solubility for different combinations of glycolipids at various pH values. For instance, ustilagic acid solubility is decreased at low pH values. Surprisingly, addition of plant-derived saponins greatly increases solubility at lower pH values and provide additional anti-microbial properties.

In a preferred embodiment the preservative method according to the present invention may include compounds increasing solubility of glycolipids selected from the group consisting of saponines obtainable from the plant genera
Quiallaja,
Yucca,
Dioscorea,
Paris,
Hosta,
Agave,
Asparagus,
Allium,
Sapindus,
Aesculus,
Hedera,
Blighia,
Dipsacus,
Aralia and/or
Anemone
   or mixtures thereof, emulgators E400 to E499 approved for food preparations in the EU, emulgators, synthetic food grade emulsifiers selected from the group consisting of lecithin; polyglycerol esters (PGE), polysorbates, stearoyl lactylates, propylene glycol esters (PGMS), sucrose esters; polyglycerol Polyricinoleate (PGPR); ammonium phosphatide (AMP); mono and diglycerides of C6-C22 saturated or unsaturated fatty acids and mixtures thereof.

### Food Preparations

Food compositions according to the invention are any preparations or compositions comprising the compositions according to the invention which are suitable for consumption and are used for nutrition or enjoyment purposes, and are generally products which are intended to be introduced into the human or animal oral cavity, to remain there for a certain time and then either be eaten (e.g. ready-to-eat foodstuffs or feeds, see also herein below) or removed from the oral cavity again (e.g. chewing gums). Such products include any substances or products which in the processed, partially processed or unprocessed state are to be ingested by humans or animals. They also include substances which are added to orally consumable products during their manufacture, preparation or treatment and which are intended to be introduced into the human or animal oral cavity.

The food compositions according to the invention also include substances which in the unchanged, treated or prepared state are to be swallowed by a human or animal and then digested; in this respect, the orally consumable products according to the invention also include casings, coatings or other encapsulations which are to be swallowed at the same time or which may be expected to be swallowed. The expression “orally consumable product” covers ready-to-eat foodstuffs and feeds, that is to say foodstuffs or feeds that are already complete in terms of the substances that are important for the taste. The expressions “ready-to-eat foodstuff” and “ready-to-eat feed” also include solid or semi-solid ready-to-eat foodstuffs or feeds. Examples which may be mentioned are frozen products, which must be thawed and heated to eating temperature before they are eaten. Products such as yoghurt or ice-cream as well as chewing gums or hard caramels are also included among the ready-to-eat foodstuffs or feeds.

Preferred food compositions according to the invention also include “semi-finished products”. Within the context of the present text, a semi-finished product is to be understood as being an orally consumable product which, because of a very high content of flavorings and taste-imparting substances, is unsuitable for use as a ready-to-eat orally consumable product (in particular foodstuff or feed). Only by mixing with at least one further constituent (e.g. by reducing the concentration of the flavorings and taste-imparting substances in question) and optionally further process steps (e.g. heating, freezing) is the semi-finished product converted into a ready-to-eat orally consumable product (in particular foodstuff or feed). Examples of semi-finished products which may be mentioned here are

Food composition according to the invention preferably comprises one or more preparations for nutrition or enjoyment purposes. These include in particular (reduced-calorie) baked goods (e.g. bread, dry biscuits, cakes, other baked articles), confectionery (e.g. chocolates, chocolate bars, other products in bar form, fruit gums, dragées, hard and soft caramels, chewing gum), meat products (e.g. ham, fresh sausage or raw sausage preparations, spiced or marinated fresh or salt meat products), eggs or egg products (dried egg, egg white, egg yolk), cereal products (e.g. breakfast cereals, muesli bars, precooked ready-to-eat rice products), dairy products (e.g. rice pudding, yoghurt, kefir, cream cheese, soft cheese, hard cheese, dried milk powder, whey, butter, buttermilk, partially or completely hydrolysed milk-protein-containing products), products made from soy protein or other soybean fractions (e.g. soy milk and products produced therefrom, preparations containing soy lecithin, fermented products such as tofu or tempeh or products produced therefrom and mixtures with fruit preparations and optionally flavors), fruit preparations (e.g. jams, sorbets, fruit sauces, fruit fillings), vegetable preparations (e.g. ketchup, sauces, dried vegetables, frozen vegetables, precooked vegetables, boiled-down vegetables), snacks (e.g. baked or fried potato crisps or potato dough products, maize- or groundnut-based extrudates), fat- and oil-based products or emulsions thereof (e.g. mayonnaise, remoulade, dressings, in each case full-fat or reduced-fat), other ready-made dishes and soups (e.g. dried soups, instant soups, precooked soups), spices, spice mixtures and in particular seasonings which are used, for example, in the snacks field, sweetener preparations, tablets or sachets, other preparations for sweetening or whitening foods. The preparations within the scope of the invention can also be used in the form of semi-finished products for the production of further preparations for nutrition or enjoyment purposes. The preparations within the scope of the invention can also be in the form of capsules, tablets (uncoated and coated tablets, e.g. enteric coatings), dragées, granules, pellets, solids mixtures, dispersions in liquid phases, in the form of emulsions, in the form of powders, in the form of solutions, in the form of pastes, or in the form of other preparations which can be swallowed or chewed, and in the form of food supplements.

The preparations can also be in the form of capsules, tablets (uncoated and coated tablets, e.g., enteric coatings), dragées, granules, pellets, solids mixtures, dispersions in liquid phases, in the form of emulsions, in the form of powders, in the form of solutions, in the form of pastes, or in the form of other preparations which can be swallowed or chewed, for example in the form of food supplements.

The semi-finished products are generally used for the production of ready-to-use or ready-to-eat preparations for nutrition or enjoyment purposes.

Further constituents of a ready-to-eat preparation or semi-finished product for nutrition or enjoyment purposes can be conventional base substances, auxiliary substances and additives for foods or enjoyment foods, for example water, mixtures of fresh or processed, vegetable or animal base or raw substances (e.g. raw, roast, dried, fermented, smoked and/or boiled meat, bone, cartilage, fish, vegetables, herbs, nuts, vegetable pastes or mixtures thereof), digestible or non-digestible carbohydrates (e.g. sucrose, maltose, fructose, glucose, dextrins, amylose, amylopectin, inulin, xylans, cellulose, tagatose), sugar alcohols (e.g. sorbitol, erythritol), natural or hardened fats (e.g. tallow, lard, palm fat, cocoa fat, hardened vegetable fat), oils (e.g. sunflower oil, groundnut oil, maize germ oil, olive oil, fish oil, soya oil, sesame oil), fatty acids or their salts (e.g. potassium stearate), proteinogenic or non-proteinogenic amino acids and related compounds (e.g. γ-aminobutyric acid, taurine), peptides (e.g. glutathione), natural or processed proteins (e.g. gelatin), enzymes (e.g. peptidases), nucleic acids, nucleotides, taste correctors for unpleasant taste impressions, further taste modulators for further, generally not unpleasant taste impressions, other taste-modulating substances (e.g. inositol phosphate, nucleotides such as guanosine monophosphate, adenosine monophosphate or other substances such as sodium glutamate or 2-phenoxypropionic acid), emulsifiers (e.g. lecithins, diacylglycerols, gum arabic), stabilisers (e.g. carrageenan, alginate), preservatives (e.g. benzoic acid and its salts, sorbic acid and its salts), antioxidants (e.g. tocopherol, ascorbic acid), chelators (e.g. citric acid), organic or inorganic acidifying agents (e.g. acetic acid, phosphoric acid), additional bitter substances (e.g. quinine, caffeine, limonene, amarogentine, humulone, lupulone, catechols, tannins), substances that prevent enzymatic browning (e.g. sulfite, ascorbic acid), ethereal oils, plant extracts, natural or synthetic colourings or colouring pigments (e.g. carotinoids, flavonoids, anthocyans, chlorophyll and derivatives thereof), spices, trigeminally active substances or plant extracts containing such trigeminally active substances, synthetic, natural or nature-identical flavourings or odorants as well as odour correctors.

Food compositions according to the invention, for example those in the form of preparations or semi-finished products, preferably comprise a flavour composition in order to complete and refine the taste and/or odour. A preparation can comprise as constituents a solid carrier and a flavour composition. Suitable flavour compositions comprise, for example, synthetic, natural or nature-identical flavourings, odorants and taste-imparting substances, reaction flavourings, smoke flavourings or other flavour-giving preparations (e.g. protein (partial) hydrolysates, preferably protein (partial) hydrolysates having a high arginine content, barbecue flavourings, plant extracts, spices, spice preparations, vegetables and/or vegetable preparations) as well as suitable auxiliary substances and carriers. Particularly suitable here are the flavour compositions or constituents thereof which produce a roasted, meaty (in particular chicken, fish, seafood, beef, pork, lamb, mutton, goat), vegetable-like (in particular tomato, onion, garlic, celery, leek, mushroom, aubergine, seaweed), spicy (in particular black and white pepper, cardamom, nutmeg, pimento, mustard and mustard products), fried, yeast-like, boiled, fatty, salty and/or pungent flavour impression and accordingly can enhance the spicy impression. The flavour compositions generally comprise more than one of the mentioned ingredients.

The food compositions of the present invention are preferably selected from the group comprising
confectionery, preferably reduced-calorie or calorie-free confectionery, preferably selected from the group comprising muesli bar products, fruit gums, dragées, hard caramels and chewing gum,
cereal products, preferably selected from the group comprising low-sugar and sugar-free breakfast cereals and muesli bars,
dairy products, preferably selected from the group comprising reduced-fat and fat-free yoghurt, kefir, whey, buttermilk and ice-cream,
products made from soy protein or other soybean fractions, preferably selected from the group comprising soy milk, products produced from soy milk, preparations containing soy lecithin, products produced from preparations containing soy lecithin and mixtures with fruit preparations and optionally flavours,
sweetener preparations, tablets and sachets,
sugar-free dragées,
ice-cream, with or without milk-based constituents, preferably sugar-free.

### Food Grade Emulsifiers

The preferred food grade emulsifiers encompass the following groups:
Lecithin;
Fatty acid derivatives, such as for example polyglycerol esters (PGE), polysorbates (“TWEEN”), stearoyl lactylates, propylene glycol esters (PGMS), and sucrose esters;
Polyglycerol Polyricinoleate (PGPR);
Ammonium Phosphatide (AMP);
Mono and Diglycerides of C₆-C₂₂ saturated or unsaturated fatty acids;
Saponins or extracts prepared from saponin-containing plants, e.g. Quillaia extract (E999)
   and mixtures thereof.

### Aroma or Flavoring Compounds

Aroma compounds and flavoring agents are well known in the art and can be chosen from synthetic flavoring liquid and/or oils derived from plants leaves, flowers, fruits and so forth, and combinations thereof. Representative flavoring liquids include: artificial, natural or synthetic fruit flavors such as eucalyptus, lemon, orange, banana, grape, lime, apricot and grapefruit oils and fruit essences including apple, strawberry, cherry, orange, pineapple and so forth; bean and nut derived flavors such as coffee, cocoa, cola, peanut, almond and so forth; and root derived flavors such as licorice or ginger.

Suitable aroma or flavoring compounds are listed in REGULATION (EC) No 1334/2008 OF THE EUROPEAN PARLIAMENT AND OF THE COUNCIL of 16 Dec. 2008 on flavorings and certain food ingredients with flavoring properties for use in and on foods and amending Council Regulation (EEC) No 1601/91, Regulations (EC) No 2232/96 and (EC) No 110/2008 and Directive 2000/13/EC.

### Sweeteners

The term “sweeteners” here denotes substances having a relative sweetening power of at least 25, based on the sweetening power of sucrose (which accordingly has a sweetening power of 1). Sweeteners to be used in an orally consumable product (in particular foodstuff, feed or medicament) according to the invention (a) are preferably non-cariogenic and/or have an energy content of not more than 5 kcal per gram of the orally consumable product.

Advantageous sweeteners in a preferred orally consumable product (in particular foodstuff, feed or medicament) according to the invention are selected from the following groups:
(a) Naturally occurring sweeteners, preferably selected from the group comprising miraculin, monellin, mabinlin, thaumatin, curculin, brazzein, pentaidin, D-phenylalanine, D-tryptophan, and extracts or fractions obtained from natural sources, comprising those amino acids and/or proteins, and the physiologically acceptable salts of those amino acids and/or proteins, in particular the sodium, potassium, calcium or ammonium salts, neohesperidin dihydrochalcone, naringin dihydrochalcone, stevioside, steviolbioside, rebaudiosides, in particular rebaudioside A, rebaudioside B, rebaudioside C, rebaudioside D, rebaudioside E, rebaudioside F, rebaudioside G, rebaudioside H, dulcosides and rubusoside, suavioside A, suavioside B, suavioside G, suavioside H, suavioside 1, suavioside J, baiyunoside 1, baiyunoside 2, phlomisoside 1, phlomisoside 2, phlomisoside 3 and phlomisoside 4, abrusoside A, abrusoside B, abrusoside C, abrusoside D, cyclocaryoside A and cyclocaryoside 1, osladin, polypodoside A, strogin 1, strogin 2, strogin 4, selligueain A, dihydroquercetin 3-acetate, perillartin, telosmoside Ais, periandrin I-V, pterocaryosides, cyclocaryosides, mukuroziocides, trans-anethole, trans-cinnamaldehyde, bryosides, bryonosides, bryonodulcosides, carnosiflosides, scandenosides, gypenosides, trilobatin, phloridzin, dihydroflavanols, hematoxylin, cyanin, chlorogenic acid, albiziasaponin, telosmosides, gaudichaudioside, mogrosides, mogroside V, hernandulcins, monatin, phyllodulcin, glycyrrhetinic acid and derivatives thereof, in particular glycosides thereof such as glycyrrhizine, and the physiologically acceptable salts of those compounds, in particular the sodium, potassium, calcium or ammonium salts; and extracts or concentrated fractions of the extracts, selected from the group comprising thaumatococcus extracts (katamfe plant), extracts from Stevia ssp. (in particular Stevia rebaudiana), swingle extracts (Momordica or Siratia grosvenorii, Luo-Han-Guo), extracts from Glycerrhyzia ssp. (in particular Glycerrhyzia glabra), extracts from Rubus ssp. (in particular Rubus suavissimus), citrus extracts and extracts from Lippia dulcis;
(b) Synthetic sweet-tasting substances, preferably selected from the group comprising magap, sodium cyclamate or other physiologically acceptable salts of cyclamic acid, acesulfame K or other physiologically acceptable salts of acesulfame, neohesperidin dihydrochalcone, naringin dihydrochalcone, saccharin, saccharin sodium salt, aspartame, superaspartame, neotame, alitame, advantame, perillartin, sucralose, lugduname, carrelame, sucrononate and sucrooctate.

### Thickeners

Advantageous thickeners in a preferred orally consumable product (in particular foodstuff, feed or medicament) according to the invention are selected from the group comprising: crosslinked polyacrylic acids and derivatives thereof, polysaccharides and derivatives thereof, such as xanthan gum, agar-agar, alginates or tyloses, cellulose derivatives, for example carboxymethylcellulose or hydroxycarboxymethylcellulose, fatty alcohols, monoglycerides and fatty acids, polyvinyl alcohol and polyvinylpyrrolidone.

Preference is given according to the invention to an orally consumable product (in particular foodstuff or feed) which comprises milk thickened with lactic acid bacteria and/or cream thickened with lactic acid bacteria and which preferably is selected from the group comprising yoghurt, kefir and quark.

A food composition according to the invention comprising milk thickened with lactic acid bacteria and/or cream thickened with lactic acid bacteria is advantageously an orally consumable product which comprises a probiotic, wherein the probiotic is preferably selected from the group comprising Bifidobacterium animalis subsp. lactis BB-12, Bifidobacterium animalis subsp. lactis DN-173 010, Bifidobacterium animalis subsp. lactis HN019, Lactobacillus acidophilus LA5, Lactobacillus acidophilus NCFM, Lactobacillus johnsonii La1, Lactobacillus casei immunitass/defensis, Lactobacillus casei Shirota (DSM 20312), Lactobacillus casei CRL431, Lactobacillus reuteri (ATCC 55730) and Lactobacillus rhamnosus (ATCC 53013).

### Additives for Chewing Gums

Particular preference is given to an orally consumable product (in particular foodstuff, feed or medicament) according to the invention that is a chewing gum and comprises a chewing-gum base. The chewing-gum base is preferably selected from the group comprising chewing-gum or bubble-gum bases. The latter are softer, so that gum bubbles can also be formed therewith. Preferred chewing-gum bases according to the invention include, in addition to the natural resins or the natural latex chicle that are traditionally used, elastomers such as polyvinyl acetate (PVA), polyethylene, (low or medium molecular weight) polyisobutene (PIB), polybutadiene, isobutene-isoprene copolymers (butyl rubber), polyvinyethyl ether (PVE), polyvinylbutyl ether, copolymers of vinyl esters and vinyl ethers, styrene-butadiene copolymers (styrene-butadiene rubber, SBR) or vinyl elastomers, for example based on vinyl acetate/vinyl laurate, vinyl acetate/vinyl stearate or ethylene/vinyl acetate, as well as mixtures of the mentioned elastomers, as described, for example, in EP 0 242 325, U.S. Pat. Nos. 4,518,615, 5,093,136, 5,266,336, 5,601,858 or U.S. Pat. No. 6,986,709. In addition, chewing-gum bases that are preferably to be used according to the invention preferably comprise further constituents such as, for example, (mineral) fillers, plasticisers, emulsifiers, antioxidants, waxes, fats or fatty oils, such as, for example, hardened (hydrogenated) vegetable or animal fats, mono-, di- or tri-glycerides. Suitable (mineral) fillers are, for example, calcium carbonate, titanium dioxide, silicon dioxide, talcum, aluminium oxide, dicalcium phosphate, tricalcium phosphate, magnesium hydroxide and mixtures thereof. Suitable plasticisers, or agents for preventing adhesion (detackifiers), are, for example, lanolin, stearic acid, sodium stearate, ethyl acetate, diacetin (glycerol diacetate), triacetin (glycerol triacetate), triethyl citrate. Suitable waxes are, for example, paraffin waxes, candelilla wax, carnauba wax, microcrystalline waxes and polyethylene waxes. Suitable emulsifiers are, for example, phosphatides such as lecithin, mono- and di-glycerides of fatty acids, for example glycerol monostearate.

Chewing gums according to the invention (in particular as disclosed above) preferably comprise constituents such as sugars of different types, sugar substitutes, other sweet-tasting substances, sugar alcohols (in particular sorbitol, xylitol, mannitol), ingredients having a cooling effect, taste correctors for unpleasant taste impressions, further taste-modulating substances (e.g. inositol phosphate, nucleotides such as guanosine monophosphate, adenosine monophosphate or other substances such as sodium glutamate or 2-phenoxypropionic acid), humectants, thickeners, emulsifiers, stabilisers, odour correctors and flavours (e.g. eucalyptus-menthol, cherry, strawberry, grapefruit, vanilla, banana, citrus, peach, blackcurrant, tropical fruits, ginger, coffee, cinnamon, combinations (of the mentioned flavours) with mint flavours as well as spearmint and peppermint on their own). The combination inter alia of the flavours with further substances that have cooling, warming and/or mouth-watering properties is of particular interest.

### Cosmetic Preparations

Another object of the present invention refers to a skin care, personal care, sun care or hair care product or product formulation, comprising the composition as defined above. The preparations may represent for example a cosmetic cream, lotion, spray, emulsion, ointment, gel or mouse and the like.

The preparations according to the invention may contain antidandruff agents, irritation-preventing agents, irritation-inhibiting agents, antioxidants, adstringents, perspiration-inhibiting agents, antiseptic agents, ant-statics, binders, buffers, carrier materials, chelating agents, cell stimulants, cleansing agents, care agents, deodorizing agents, antiperspirants, softeners, emulsifiers, enzymes, essential oils, fibres, film-forming agents, fixatives, foam-forming agents, foam stabilizers, substances for preventing foaming, foam boosters, gelling agents, gel-forming agents, hair care agents, hair-setting agents, hair-straightening agents, moisture-donating agents, moisturizing substances, moisture-retaining substances, bleaching agents, strengthening agents, stain-removing agents, optically brightening agents, impregnating agents, dirt-repellent agents, friction-reducing agents, lubricants, moisturizing creams, ointments, opacifying agents, plasticizing agents, covering agents, polish, gloss agents, polymers, powders, proteins, re-oiling agents, abrading agents, silicones, hair promotion agents, cooling agents, skin-cooling agents, warming agents, skin-warming agents, stabilizers, UV-absorbing agents, UV filters, detergents, thickeners, vitamins, oils, waxes, fats, phospholipids, saturated fatty acids, mono- or polyunsaturated fatty acids, α-hydroxy acids, polyhydroxyfatty acids, liquefiers, dyestuffs, color-protecting agents, pigments, odoriferous substances, polyols, surfactants, electrolytes, organic solvents or silicone derivatives and the like as additional auxiliaries and additives.

### INDUSTRIAL APPLICATION

The present invention also encompasses the use of glycolipids as antimicrobial agents against at least one of the following micro-organisms
Asaia siamensis
Bacillus licheniformis
Bacillus subtilis
Byssochlamys fulva
Byssochlamys nivea
Candida parapsilosis
Clostridium perfringens
Clostridium sporogenes
Corynebacterium xerosis
Enterococcus faecalis
Lactobacillus paracasei ssp. paracasei
Leuconostoc lactis
Leuconostoc mesenteroides
Malassezia furfur
Neosartorya fischeri
Penicillium paneum
Saccharomyces cerevisiae
Yarrowia lipolytica
Zygosaccharomyces bailii
   for preserving compositions at a pH of about 1 to about 4, wherein said glycolipid represents an ustilagic acid or an ester of ustilagic acid esterified with a linear C₁-C₄ aliphatic alcohol.

In a preferred embodiment the said at least one ustilagic acid or its ester is added in a working amount of from about 1 to about 1,000 ppm—calculated on the composition.

In a preferred embodiment the said composition is a cosmetic, detergent or food preparation excluding beverages.

For the sake of good order, it is emphasized that all preferred embodiments mentioned above, particularly with respect to combinations, ratios and amounts also apply to the method and the use as claimed. Therefore, their repetition is not necessary.

### Examples

### Manufacturing Examples

### Fermentation

The strain Ustilago maydis 04507fxxx000001 was revived on 2% malt extract agar after storage at −80° C. As seed flasks 500 ml-Erlemnmeyer flasks with 2 baffles and closed with PU-foam bungs were used. Each flask containing 100 ml seed medium (malt extract 3%; Tween 85 0.2%; agar agar 0.1%; pH adjusted to pH 7.0) was inoculated with 3-4 agar pieces from the revival plates. The flasks were incubated for 48 hours at 25° C. on an orbital shaker with 50 mm shaking orbit. The main culture was inoculated at a ratio of 4% seed. For the main culture a fermenter of 12 L volume was filled with 9 L of a production medium containing dextrose 5%; ammonium sulfate 0.13%; potassiumdi-hydrogenphosphate 0.25%; magnesium sulfate heptahydrate 0.13%; sodium chloride 0.1%; calcium chloride dihydrate 0.1%; vitamin solution RPMI 1640 (Sigma-Aldrich) 1 ml/I and a trace element solution in tap water with the pH-value adhusted to pH 6.5. The trace element solution provided ferrous sulfate 5.4 μM; zinc sulfate 3.1 μM; manganese sulfate 2.4 μM; copper(II) sulfate 2.2 μM; cobalt nitrate 2.1 μM; boric acid 4.0 μM; and sodium molybdate 0.8 μM. Fermentation conditions were incubation at 25° C., stirring at 350 rpm with two Rushton type stirrers, aeration at 0.7 vvm and foam control with a silicon based antifoam compound. After 120 hours of fermentation the culture was harvested by centrifugation. After centrifugation for 25 minutes with 10,000 rcf biomass and ustilagic acid were sedimented.

### Extraction and Isolation of Glycolipids

### Preparation of the Raw Extract

After harvest the ustilagic acids were contained in the centrifugation sediment together with the biomass from the fermentation. This sediment was frozen, lyophilized and extracted with warm ethanol. After evaporation of the solvent the raw extract was received.

### Pre-Fractionation by Reverse Phase Chromatography

The raw <extract was subjected to a reverse phase medium pressure chromatography to enrich the compounds of interest. Reverse phase MPLC allows to remove sugars and lipids as well as other secondary metabolites like flavonoids very efficiently and to reach enriched fractions of compounds of interest, which could be put on a second fractions step by HPLC using finer RP material. Sometimes it makes sense, to combine different MPLC prefractionations methods to get a more specific enrichment of selected saponins, e.g., by combination of RP18 and RP4 material.

### Second Purification Step by Reverse Phase Chromatography

Pure compounds were isolated by reverse phase chromatography using different fractions from the first separation step by MPLC. To reach a purity of >90% per compound sometimes several repeated separations by HPLC were necessary. Promising fractions from the preparative HPLC runs detected by light scattering detection ELSD were analyzed by LCMS. The solvent of all fractions containing compounds of interest was removed in vacuum followed by a freeze-drying step. Isolated compounds were characterized by LCMS and 1 D and 2D NMR spectroscopy.

### Isolated Compounds

TABLE 1
Isolated compounds

| ID | Compound | Structure |
|---|---|---|
| NP- 018245 | | 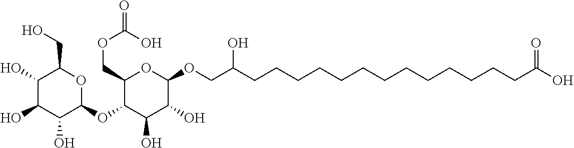 [SMILES: O=C(O)CCCCCCCCCCCCCC(O)CO[C@@H]1O[C@H](COC(=O)O)[C@@H](O[C@@H]2O[C@H](CO)[C@@H](O)[C@H](O)[C@H]2O)[C@H](O)[C@H]1O] |
| NP- 018428 | | 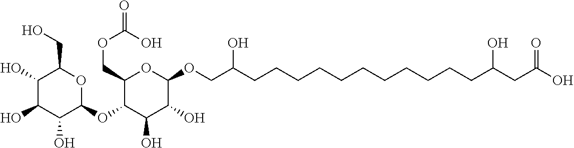 [SMILES: O=C(O)CC(O)CCCCCCCCCCCC(O)CO[C@@H]1O[C@H](COC(=O)O)[C@@H](O[C@@H]2O[C@H](CO)[C@@H](O)[C@H](O)[C@H]2O)[C@H](O)[C@H]1O] |
| NP- 018243 | | 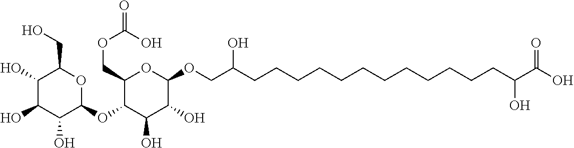 [SMILES: O=C(O)OC[C@H]1O[C@@H](OCC(O)CCCCCCCCCCCCC(O)C(=O)O)[C@H](O)[C@@H](O)[C@@H]1O[C@@H]1O[C@H](CO)[C@@H](O)[C@H](O)[C@H]1O] |
| NP- 018247 | | 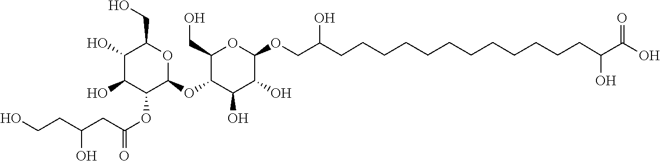 [SMILES: O=C(CC(O)CCO)O[C@H]1[C@H](O[C@H]2[C@H](O)[C@@H](O)[C@H](OCC(O)CCCCCCCCCCCCC(O)C(=O)O)O[C@@H]2CO)O[C@H](CO)[C@@H](O)[C@@H]1O] |
| NP- 018486 | 5 | 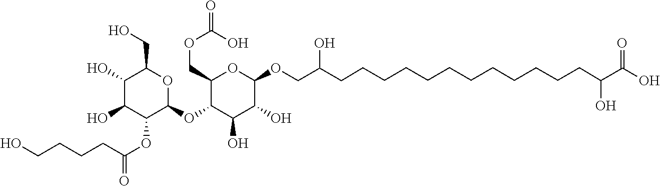 [SMILES: O=C(O)OC[C@H]1O[C@@H](OCC(O)CCCCCCCCCCCCC(O)C(=O)O)[C@H](O)[C@@H](O)[C@@H]1O[C@@H]1O[C@H](CO)[C@@H](O)[C@H](O)[C@H]1OC(=O)CCCCO] |
| NP- 018255 | | 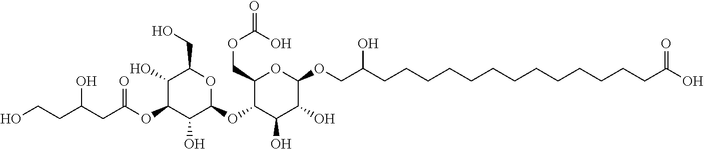 [SMILES: O=C(O)CCCCCCCCCCCCCC(O)CO[C@@H]1O[C@H](COC(=O)O)[C@@H](O[C@@H]2O[C@H](CO)[C@@H](O)[C@H](OC(=O)CC(O)CCO)[C@H]2O)[C@H](O)[C@H]1O] |
| NP- 018250 | 3 | 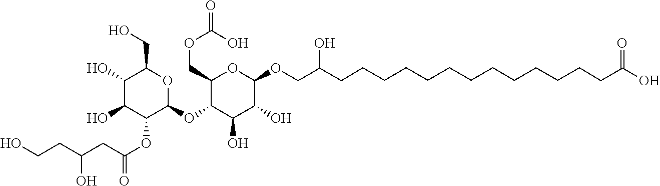 [SMILES: O=C(O)CCCCCCCCCCCCCC(O)CO[C@@H]1O[C@H](COC(=O)O)[C@@H](O[C@@H]2O[C@H](CO)[C@@H](O)[C@H](O)[C@H]2OC(=O)CC(O)CCO)[C@H](O)[C@H]1O] |
| NP- 018756 | | 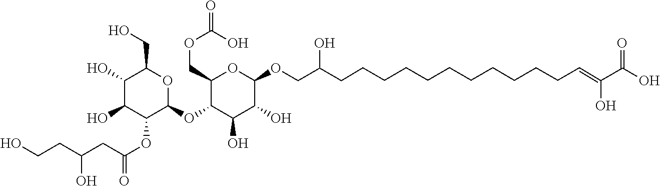 [SMILES: O=C(O)OC[C@H]1O[C@@H](OCC(O)CCCCCCCCCCC/C=C(\O)C(=O)O)[C@H](O)[C@@H](O)[C@@H]1O[C@@H]1O[C@H](CO)[C@@H](O)[C@H](O)[C@H]1OC(=O)CC(O)CCO] |
| NP- 018249 | | 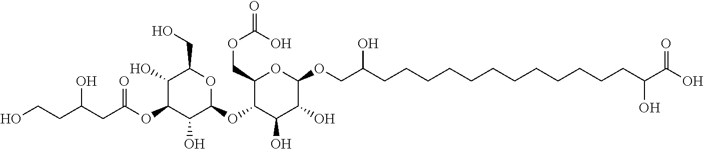 [SMILES: O=C(O)OC[C@H]1O[C@@H](OCC(O)CCCCCCCCCCCCC(O)C(=O)O)[C@H](O)[C@@H](O)[C@@H]1O[C@@H]1O[C@H](CO)[C@@H](O)[C@H](OC(=O)CC(O)CCO)[C@H]1O] |
| NP- 018459 | | 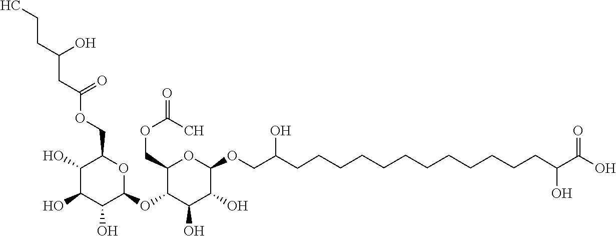 [SMILES: CCCC(O)CC(=O)OC[C@H]1O[C@@H](O[C@H]2[C@H](O)[C@@H](O)[C@H](OCC(O)CCCCCCCCCCCCC(O)C(=O)O)O[C@@H]2COC(C)=O)[C@H](O)[C@@H](O)[C@@H]1O] |
| NP- 018217 | 1 | 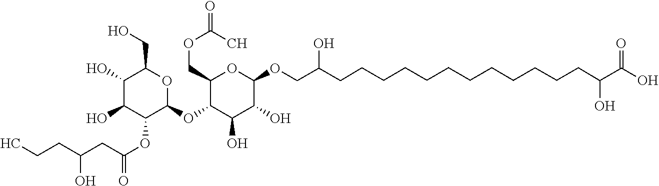 [SMILES: CCCC(O)CC(=O)O[C@H]1[C@H](O[C@H]2[C@H](O)[C@@H](O)[C@H](OCC(O)CCCCCCCCCCCCC(O)C(=O)O)O[C@@H]2COC(C)=O)O[C@H](CO)[C@@H](O)[C@@H]1O] |
| NP- 018752 | 4 | 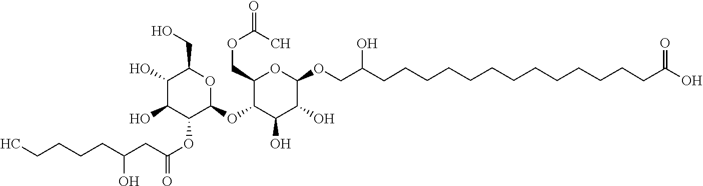 [SMILES: CCCCCC(O)CC(=O)O[C@H]1[C@H](O[C@H]2[C@H](O)[C@@H](O)[C@H](OCC(O)CCCCCCCCCCCCCC(=O)O)O[C@@H]2COC(C)=O)O[C@H](CO)[C@@H](O)[C@@H]1O] |
| NP- 018753 | 6 | 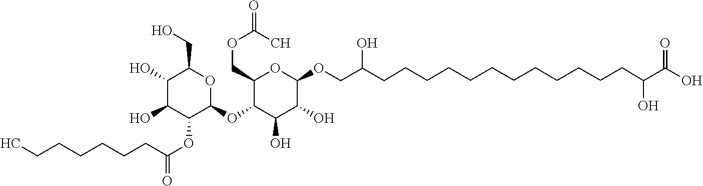 [SMILES: CCCCCCCC(=O)O[C@H]1[C@H](O[C@H]2[C@H](O)[C@@H](O)[C@H](OCC(O)CCCCCCCCCCCCC(O)C(=O)O)O[C@@H]2COC(C)=O)O[C@H](CO)[C@@H](O)[C@@H]1O] |
| NP- 018218 | | 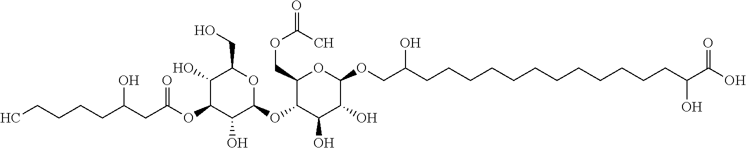 [SMILES: CCCCCC(O)CC(=O)O[C@@H]1[C@@H](O)[C@H](O[C@H]2[C@H](O)[C@@H](O)[C@H](OCC(O)CCCCCCCCCCCCC(O)C(=O)O)O[C@@H]2COC(C)=O)O[C@H](CO)[C@H]1O] |
| NP- 018256 | 2 | 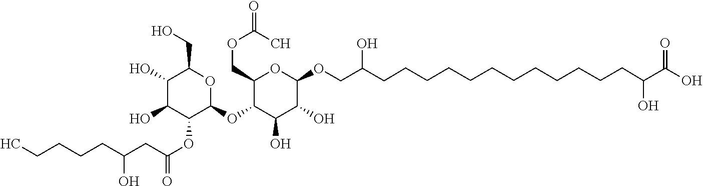 [SMILES: CCCCCC(O)CC(=O)O[C@H]1[C@H](O[C@H]2[C@H](O)[C@@H](O)[C@H](OCC(O)CCCCCCCCCCCCC(O)C(=O)O)O[C@@H]2COC(C)=O)O[C@H](CO)[C@@H](O)[C@@H]1O] |
| NP- 018430 | | 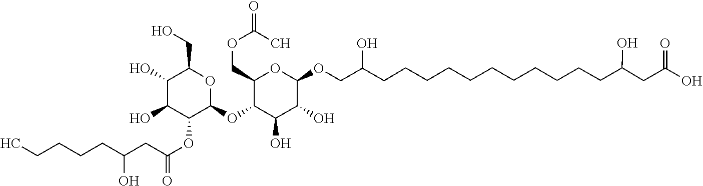 [SMILES: CCCCCC(O)CC(=O)O[C@H]1[C@H](O[C@H]2[C@H](O)[C@@H](O)[C@H](OCC(O)CCCCCCCCCCCC(O)CC(=O)O)O[C@@H]2COC(C)=O)O[C@H](CO)[C@@H](O)[C@@H]1O] |
| NP- 018432 | | 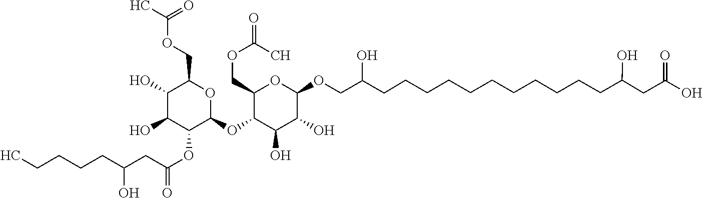 [SMILES: CCCCCC(O)CC(=O)O[C@H]1[C@H](O[C@H]2[C@H](O)[C@@H](O)[C@H](OCC(O)CCCCCCCCCCCC(O)CC(=O)O)O[C@@H]2COC(C)=O)O[C@H](COC(C)=O)[C@@H](O)[C@@H]1O] |
| NP- 018431 | | 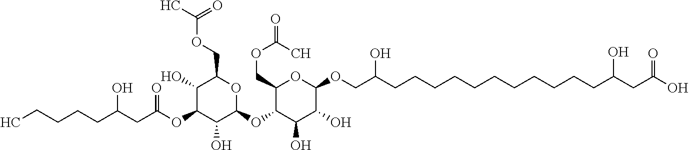 [SMILES: CCCCCC(O)CC(=O)O[C@@H]1[C@@H](O)[C@H](O[C@H]2[C@H](O)[C@@H](O)[C@H](OCC(O)CCCCCCCCCCCC(O)CC(=O)O)O[C@@H]2COC(C)=O)O[C@H](COC(C)=O)[C@H]1O] |

Isolated compounds

| ID | Formula | CAS |
|---|---|---|
| NP- | C30H54O15 | 1427028-85-0 |
| 018245 | | |
| NP- | C30H54O16 | 1198760-80-3 |
| 018428 | | |
| NP- | C30H54O16 | 1427028-84-9 |
| 018243 | | |
| NP- | C34H62O17 | |
| 018247 | | |
| NP- | C36H64O17 | |
| 018486 | | |
| NP- | C36H64O17 | 1427028-86-1 |
| 018255 | | |
| NP- | C36H64O17 | 1427028-86-1 |
| 018250 | | |
| NP- | C36H62O18 | |
| 018756 | | |
| NP- | C36H64O18 | 1427087-15-7 |
| 018249 | | |
| NP- | C36H64O18 | 959757-52-9 |
| 018459 | | |
| NP- | C36H64O18 | 867578-35-6 |
| 018217 | | |
| NP- | C38H68O17 | |
| 018752 | | |
| NP- | C38H68O17 | 1427039-33-5 |
| 018753 | | |
| NP- | C38H68O18 | 1427087-13-5 |
| 018218 | | |
| NP- | C38H68O18 | 1427087-16-8 |
| 018256 | | |
| NP- | C38H68O18 | 1199256-46-6 |
| 018430 | | |
| NP- | C40H70O19 | 1427113-19-6 |
| 018432 | | |
| NP- | C40H70O19 | |
| 018431 | | |

### Antimicrobial Activity

Compounds are dissolved at a concentration of 20 mg/mL using 100% DMSO.
Preculture: yeast and bacteria are plated on potato dextrose agar, resuspended in potato dextrose broth and CFU via OD measurement determined; adjusting CFU of the strain in PDB pH 3.8
Preculture: for mycel forming fungi spores were directly used after thawing of cryopreserved stocks of known titre
Final titre used for the assay: Yeast strains 5*10³ CFU/ml, Bacteria 1*10⁶ CFU/ml, Fungi (spores) 5*10⁴ CFU/ml
Potato dextrose broth pH 3.8 was used as assay matrix
500 ppm (0.05%) of the compounds were screened (final DMSO 2.5% (v/v))
Volume: 100 μl in 96-well format
Positive controls: potassium sorbate, sodium benzoate & triclosan
28° C. to 30° C., static incubation
Incubation time depending on spoilage organism between 2 and 8 days
Visual evaluation (per eye, for fungi with binocular magnification)

TABLE 2
List of strains

| Class | Taxon | Strain no. |
|---|---|---|
| yeast | Saccharomyces cerevisiae | DSM 2548 |
| | Zygosaccharomyces bailii | SUN02 |
| | Candida albicans | ATCC10231 |
| | Candida parapsilosis | SUN05 |
| | Saccharomyces cerevisiae | DSM 2548 |
| | Zygosaccharomyces bailii | DSM 70834 |
| | Brettanomyces sp | SUN08 |
| | Brettanomyces naardensis | CBS 6115 |
| | Zygosaccharomyces rouxii | DSM 7525 |
| | Yarrowia lipolytica | DSM 70561 |
| bacteria | Asaia siamensis | SUN03 |
| | Alicyclobacillus acidoterrestris | DSM 2498 |
| | Bacillus licheniformis | DSM 13 |
| | Bacillus subtilis | ATCC 6633 |
| | Clostridium perfringens | DSM756 |
| | Clostridium sporogenes | ATCC19404 |
| | Corynebacterium xerosis | DSM 20743 |
| | Enterococcus faecalis | ATCC 7080 |
| | Gluconacetobacter liquefaciens | DSM 5603 |
| | Lactiplantibacillus plantarum ssp. plantarum | DSM 20174 |
| | Lactobacillus paracasei ssp. paracasei | DSM 46331 |
| | Leuconostoc lactis | DSM 20202 |
| | Leuconostoc mesenteroides | DSM 20343 |
| fungi | Penicillium paneum | SUN04 |
| | Neosartorya fisheri | SUN09 |
| | Byssochlamys nivea | SUN10 |
| | Byssochlamys fulva | DSM 1808 |
| | Aspergillus brasiliensis | DSM 1988 |
| | Penicillium expansum | DSM 62841 |
| | Malassezia furfur | DSM 6170 |
| | Dekkera naardenensis | CBS 6115 |

TABLE 3
antimicrobial activities (Minimal Inhibitory Concentration (MIC) in μg/ml) of tested compounds
(color-coded columns: blue: compounds specifically claimed in claim 6 of this application)

| Species | Strain | NP-018217 | NP-018218 | NP-018243 | NP-018249 | NP-018250 | NP-018256 | NP-018428 |
|---|---|---|---|---|---|---|---|---|
| Asaia siamensis | SUN03 | 500 | inact. | inact. | inact. | ≤125 | inact. | inact. |
| Alicyclobacillus | DSM 2498 | 240 | | | | | 240 | |
| acidoterrestris | | | | | | | | |
| Aspergillus brasiliensis | DSM 1988 | | | | | | 240 | |
| Bacillus licheniformis | DSM 13 | | | | | | 240 | |
| Bacillus subtilis | ATCC 6633 | 600 | | | | 250 | | |
| Brettanomyces sp | SUN08 | ≤125 | inact. | 250 | inact. | 125 | ≤125 | inact. |
| Byssochlamys fulva | DSM 1808 | 80 | | | | | | |
| Byssochlamys nivea | SUN10 | ≤31.25 | inact. | 250 | inact. | 125 | ≤125 | inact. |
| Candida albicans | ATCC10231 | 80 | | | | | 80 | |
| Candida parapsilosis | ATCC 20384 | | | | | | 240 | |
| Candida parapsilosis | SUN05 | ≤125 | inact. | 500 | inact. | 125 | ≤125 | inact. |
| Clostridium perfringens | DSM756 | | | | | | 80 | |
| Clostridium sporogenes | ATCC19404 | 80 | | | | 250 | 80 | |
| Corynebacterium | DSM 20743 | 240 | | | | | 1200 | |
| xerosis | | | | | | | | |
| Dekkera naardenensis | CBS 6115 | 240 | | | | | 80 | |
| Enterococcus faecalis | ATCC 7080 | 240 | | | | 250 | | |
| Gluconacetobacter | DSM 5603 | | | | | 250 | | |
| liquefaciens | | | | | | | | |
| Lactobacillus paracasei | DSM 46331 | 80 | | | | | 240 | |
| ssp. paracasei | | | | | | | | |
| Leuconostoc lactis | DSM 20202 | 250 | | | | 250 | 240 | |
| Leuconostoc | DSM 20343 | 240 | | | | | 240 | |
| mesenteroides | | | | | | | | |
| Malassezia furfur | DSM 6170 | 80 | | | | | 80 | |
| Neosartorya fischeri | SUN09 | ≤31.25 | inact. | ≤125 | inact. | 125 | ≤125 | 500 |
| Penicillium expansum | DSM 62841 | 240 | | | | | 240 | |
| Penicillium paneum | SUN04 | ≤31.25 | inact. | ≤125 | inact. | 125 | ≤125 | inact. |
| Saccharomyces | DSM 2548 | 240 | | | | | 240 | |
| cerevisiae | | | | | | | | |
| Saccharomyces | SUN01 | ≤125 | inact. | 250 | 500 | 125 | ≤125 | inact. |
| cerevisiae | | | | | | | | |
| Saccharomyces | SUN06 | ≤125 | 500 | 250 | inact. | 125 | ≤125 | inact. |
| cerevisiae | | | | | | | | |
| Yarrowia lipolytica | DSM 70561 | 240 | | | | | 240 | |
| Zygosaccharomyces | DSM 70834 | 80 | | | | | 80 | |
| bailii | | | | | | | | |
| Zygosaccharomyces | SUN02 | ≤125 | inact. | 250 | 500 | 125 | ≤125 | inact. |
| bailli | | | | | | | | |
| Zygosaccharomyces | SUN07 | 500 | inact. | 250 | 500 | 125 | 500 | inact. |
| bailli | | | | | | | | |
| Zygosaccharomyces | DSM 7525 | 240 | | | | | 80 | |
| rouxii | | | | | | | | |

| Species | Strain | NP-018459 | NP-018752 | NP-018753 | NP-018756 | NP-019339 | NP-019340 |
|---|---|---|---|---|---|---|---|
| Asaia siamensis | SUN03 | inact. | inact. | >500 | >500 | inact. | inact. |
| Alicyclobacillus | DSM 2498 | | 4 | | 16 | | |
| acidoterrestris | | | | | | | |
| Aspergillus brasiliensis | DSM 1988 | | | | | | |
| Bacillus licheniformis | DSM 13 | | | | | | |
| Bacillus subtilis | ATCC 6633 | | | | 600 | | |
| Brettanomyces sp | SUN08 | inact. | inact. | 500 | 250 | inact. | inact. |
| Byssochlamys fulva | DSM 1808 | | | | | | |
| Byssochlamys nivea | SUN10 | inact. | 500 | ≤31.25 | ≤125 | inact. | inact. |
| Candida albicans | ATCC10231 | | | | | | |
| Candida parapsilosis | ATCC 20384 | | | | | | |
| Candida parapsilosis | SUN05 | inact. | inact. | ≤31.25 | 500 | inact. | inact. |
| Clostridium perfringens | DSM756 | | | | | | |
| Clostridium sporogenes | ATCC19404 | | | | | | |
| Corynebacterium | DSM 20743 | | | | | | |
| xerosis | | | | | | | |
| Dekkera naardenensis | CBS 6115 | | | | | | |
| Enterococcus faecalis | ATCC 7080 | | | | | | |
| Gluconacetobacter | DSM 5603 | | | | | | |
| liquefaciens | | | | | | | |
| Lactobacillus paracasei | DSM 46331 | | | | | | |
| ssp. paracasei | | | | | | | |
| Leuconostoc lactis | DSM 20202 | | | | 600 | | |
| Leuconostoc | DSM 20343 | | | | | | |
| mesenteroides | | | | | | | |
| Malassezia furfur | DSM 6170 | | | | | | |
| Neosartorya fischeri | SUN09 | 500 | inact. | ≤31.25 | ≤125 | inact. | inact. |
| Penicillium expansum | DSM 62841 | | | | | | |
| Penicillium paneum | SUN04 | 500 | inact. | 500 | 250 | inact. | inact. |
| Saccharomyces | DSM 2548 | | | | | | |
| cerevisiae | | | | | | | |
| Saccharomyces | SUN01 | inact. | inact. | ≤31.25 | 250 | inact. | inact. |
| cerevisiae | | | | | | | |
| Saccharomyces | SUN06 | inact. | 500 | ≤31.25 | 250 | inact. | inact. |
| cerevisiae | | | | | | | |
| Yarrowia lipolytica | DSM 70561 | | | | | | |
| Zygosaccharomyces | DSM 70834 | | | | | | |
| bailii | | | | | | | |
| Zygosaccharomyces | SUN02 | inact. | 500 | ≤31.25 | 250 | inact. | inact. |
| bailli | | | | | | | |
| Zygosaccharomyces | SUN07 | 500 | 500 | ≤31.25 | 250 | inact. | inact. |
| bailli | | | | | | | |
| Zygosaccharomyces | DSM 7525 | | | | | | |
| rouxii | | | | | | | |

The examples clearly demonstrate that the glycolipids according to the present invention exhibit a serious activity against a wide spectrum of microorganisms, which typically can be found in products of daily life as foodstuff, petfood or cosmetics. The performance of glycolipids according to the present invention is comparable to sorbic and benzoic acid, common natural preservatives, and can be synergistically increased either by blending two or more glycolipids according to the invention or by using blends of said glycolipids with secondary preservatives like sorbic acid, benzoic acid, lactic acid, ethanol or PHB (esters).

### FORMULATION EXAMPLES

### Formulation Examples Food

The following examples F1 to F2 show various formulations for food preparations.

Glycolipid stands for one or more glycolipids according to Claim 4.

### Example F1

An Italian Salad dressing formulation is prepared as follows:

| Ingredient | Parts |
|---|---|
| Part I | |
| water | 70.0 |
| Vinegar | 7.0 |
| Sodium Chloride | 2.0 |
| Sweetener | 4.0 |
| Gum | 0.4 |
| Flavors | 0.9 |
| Part II | |
| Vegetable Oil | 5.3 |
| Part III | |
| Water | 6.2 |
| Vinegar | 4.2 |
| Spices and Flavors | 1.8 |

Parts 1 and 2 are mixed and homogenized. Part 3 is then added and thoroughly mixed to form a homogenous mixture. 400 ppm of glycolipid is added to the dressing formula.

### Example F2: Ketchup

### Aqueous Phase

| | |
|---|---|
| ground tomato | 25.9 |
| cider vinegar | 20.7 |
| water | 44.3 |
| Sundried tomatoes | 2.1 |
| sodium chloride | 1.8 |
| dehydrated minced garlic | 1.1 |
| frozen oregano | 0.4 |
| basil | 0.5 |
| Xanthan gum | 0.2 |
| white onion powder | 2.5 |
| sucrose | 7.0 |
| EDTA | 0.0007 |
| glycolipid | 0.05 |

### Oil Phase

| | |
|---|---|
| soybean oil | 53.3 |
| olive oil | 46.7 |

### Formulation Examples Petfood

The following examples P1 to P4 show various formulations for pet food preparations. Glycolipid stands for one or more glycolipids according to Claim 4.

### Example P1

This example describes the preparation of a 60% moisture, microbiologically-stable food product simulating meat chunks which is suitable for use as a dog food. The food is prepared from the following ingredients:

| Ingredient | Parts |
|---|---|
| Wheat gluten | 10.0 |
| Soya bean oil meal | 4.0 |
| Poultry meal | 3.0 |
| Meat meal | 1.5 |
| Corn meal | 1.2 |
| Tallow | 5.0 |
| Carrageenan | 2.5 |
| Potassium sorbate | 0.3 |
| Tween 80 | 0.25 |
| Span 80 | 0.75 |
| Glycolipid | 0.5 |
| High fructose corn syrup | 15.0 |
| (80% solids, 90% fructose) | |
| Water | 56.0 |

To prepare the food, the carrageenan is added to the water at 70° C. and stirred to dissolve. The Tween 80, polyoxyethylene sorbitan monoleate available from Atlas Chemical, potassium sorbate, benzyl alcohol and the high fructose corn syrup (Isomerose 900, available from Clinton Corn Products) are added to the carrageenan solution and stirred vigorously to dissolve. The tallow is melted and the Span 80, sorbitan monooleate, available from Atlas Chemical, is added to it and mixed thoroughly. The tallow and aqueous solution are then mixed vigorously to emulsify. The remaining dry components are added with stirring to blend with the emulsion. The finished mix is added to a pan and autoclaved at 15 lbs gauge pressure for 20 minutes to heat set the mass. The heat-set cohesive product has a moisture content of 60% and is microbiologically stable. Without the glycolipid component this formulation would not be microbiologically stable.

### Example P2

This example describes the production of a 70% moisture, microbiologically stable food product simulating meat chunks from the following ingredients:

| Ingredient | Parts |
|---|---|
| Wheat gluten | 7.48 |
| Soya bean oil meal | 2.96 |
| Poultry meal | 2.23 |
| Meat meal | 1.11 |
| Corn meal | 0.88 |
| Tallow | 3.70 |
| Carrageenan | 1.85 |
| Potassium sorbate | 0.30 |
| Span 80 | 0.75 |
| Tween 80 | 0.25 |
| Glycolipid | 0.50 |
| High fructose corn syrup | 10.10 |
| Water | 66.90 |

The process is the same as in Example 1.

### Example P3

This example describes the preparation of a microbiologically-stable gravy having moisture content of 60%. The formulation is as follows:

| Ingredient | Parts |
|---|---|
| Meat & bone meal | 9.45 |
| Yeast | 1.35 |
| Corn flour | 1.35 |
| Dried whey | 1.35 |
| Whole egg solids | 1.35 |
| Tallow | 9.45 |
| High fructose corn syrup | 20.23 |
| Tween 80 | 0.25 |
| Span 80 | 0.75 |
| Glycolipid | 0.50 |
| Kelcosol | 0.14 |
| Water | 54.0 |

The process according to this example calls for first mixing the Span 80 with the tallow in melted condition. Next, a solution containing the high fructose corn syrup, water, potassium sorbate, Tween 80, kelcosol, sodium alginate, available from Kelco Co., and benzyl alcohol is prepared by heating the water to 60° C. and mixing. The solution is then stirred vigorously with the tallow to emulsify. The remaining dry materials are then added and stirred vigorously. No heating is necessary other than in the initial mixing of the fat and aqueous phases.

### Example P4

This example describes the preparation of a microbiologically-stable gravy having a moisture content of 70%. The following ingredients are combined according to the process of Example 3:

| Ingredient | Parts |
|---|---|
| Meat & bone meal | 7.0 |
| Corn flour | 1.0 |
| Brewers yeast | 1.0 |
| Dried whey | 1.0 |
| Whole egg solids | 1.0 |
| Tallow | 7.0 |
| High fructose corn syrup | 15.0 |
| Kelcosol | 0.1 |
| Span 80 | 0.75 |
| Tween 80 | 0.25 |
| Glycolipid | 0.50 |
| Water | 65.30 |

### Formulation Examples Cosmetics & Detergents

The following examples F1 to F56 show various formulations for preparations. Glycolipid stands for one or more glycolipids according to Claim 4.

TABLE F1
Liquid soap; transparent (Amounts in % b.w.)

| Ingredients | INCI Name | Amount |
|---|---|---|
| Tagat O 2 | PEG-20 Glyceryl Oleate | 2.5 |
| Coconut oil diethanolamine | Cocamide DEA | 5.0 |
| condensate | | |
| Abil B 8842 | Cyclomethicone | 0.5 |
| Sodium laurylethersulfate. 28% | Sodium Laureth Sulfate | 35.0 |
| Tego-Betaine L7 | Cocamidopropyl Betaine | 5.0 |
| Soap. 25% | Coconut acid. Potassium | 20.0 |
| | salt. Potassium Oleate | |
| Hydrolite ® 5 | 1,2-propanediol | 0.4 |
| Glycolipids | | 0.3 |
| Water | Water | Ad 100 |

TABLE F2
Syndet soap; liquid (Amounts in % b.w.)

| Ingredients | INCI Name | Amount |
|---|---|---|
| Elfan OS 46 | Sodium Olefin C14-C16 Sulfonate | 35.5 |
| Armoteric LB | Lauryl Betaine | 8.0 |
| Elfan SG | | 10.0 |
| Elfacos GT 282 L | Talloweth-60 Myristyl Glycol | 3.0 |
| PCL-Liquid 100 | Cetearyl Ethylhexanoate | 4.0 |
| Hydrolite ® 5 Green | Hydrolite ® 5 Green | 0.4 |
| Glycolipids | | 0.4 |
| Water | Water | Ad 100 |

TABLE F3
Cosmetic lotion for body wash (Amounts in % b.w.)

| Ingredients | INCI Name | Amount |
|---|---|---|
| Lumerol K 28 | Disodium Laureth Sulfosuccinate. | 33.0 |
| | Cocamidopropyl Betaine. Magnesium | |
| | Lauryl Sulfate | |
| Amphotensid B 4 | Cocamidopropyl Betaine | 10.0 |
| Perlglanzmittel | MIPA-Pareth-25 Sulfate. Glycol | 4.0 |
| GM 4055 | Stearate | |
| Sodium Chloride | Sodium Chloride | 2.0 |
| Avocado oil | Persea Gratissima (Avocado) Oil | 3.0 |
| Water | Water | Ad 100 |
| Hydrolite ® 5 | Hydrolite ® 5 Green | 0.5 |
| Euxyl ® K727 | Phenoxyethanol. Methyldibromo | 0.3 |
| | Glutaronitrile. Isothiazolinones | |
| Glycolipids | | 0.6 |

TABLE F4
Cosmetic lotion for body wash with Triclosan (Amounts in % b.w.)

| Ingredients | INCI Name | Amount |
|---|---|---|
| Texapon N 25 | Sodium Laureth Sulfate | 37.5 |
| Lamepon S | Potassium Cocoyl Hydrolyzed Collagen | 28.0 |
| Lamesoft LMG | Hydrogenated Tallow Glycerides. TEA- | 5.0 |
| | Cocoyl Hydrolyzed Collagen | |
| Lamesoft 156 | Glyceryl Laurate. TEA-Cocoyl | 5.0 |
| | Hydrolyzed Collagen | |
| Sodium Chloride | Sodium Chloride | 1.7 |
| Irgasan DP 300 | Triclosan | 0.5 |
| Water | Water | Ad 100 |
| Hydrolite ® 5 | Hydrolite ® 5 Green | 0.3 |
| Glycolipids | | 0.4 |

TABLE F5
Intimate wash (Amounts in % b.w.)

| Ingredients | INCI Name | Amount |
|---|---|---|
| Tegobetaine HS | Cocamidopropyl Betaine. Glyceryl | 15.0 |
| | Laurate | |
| Tagat L 2 | PEG-20 Glyceryl Laurate | 2.0 |
| Arlacide G | Chlorhexidine Digluconate | 0.1 |
| Rewoquat B 50 | Benzalkonium Chloride | 0.1 |
| Lactic Acid. 80% | Lactic Acid | 0.1 |
| Water | Water | Ad 100 |
| Hydrolite ® 5 | Hydrolite ® 5 Green | 0.2 |
| Euxyl ® K700 | Potassium Sorbate. Benzyl Alcohol. | 0.3 |
| | Phenoxyethanol | |
| Glycolipids | | 0.5 |

TABLE F6
Liquid soap (Amounts in % b.w.)

| Ingredient | INCI | Amount |
|---|---|---|
| Deionized water | Water | 2.0 |
| Soap bases mix | Sodium tallowates/palmitates | 95.8 |
| Titanium dioxide | Titanium dioxide | 1.0 |
| Hydrolite ® 5 | Hydrolite ® 5 Green | 1.2 |
| Preservatives | Phenoxyethanol | 0.5 |
| Glycolipids | | 0.5 |

TABLE F7
Shampoo (Amounts in % b.w.)

| Ingredients | Amount |
|---|---|
| Sodium lauryl ether sulfate (e.g. Texapon NSO) | 12 |
| Cocamidopropyl betaine (e.g. Dehyton K) | 2 |
| Sodium chloride | 1.4 |
| Citric acid | 1.3 |
| 1,2-pentanediol (Hydrolite ® 5) | 0.3 |
| Phenoxyethanol. methyl-. ethyl-. butyl- and | 0.5 |
| propylparaben | |
| Glycolipids | 0.5 |
| Water | Ad 100 |

TABLE F8
2-in-1 Shampoo (Amounts in % b.w.)

| Ingredients | INCI Name | Amount |
|---|---|---|
| Deionized water | Water | Ad 100 |
| Plantacare PS 10 | Sodium Laureth Sulfate. Lauryl | 20.0 |
| | Glucoside | |
| Euperlan PK 771 | Glycol Distearate. Sodium Lauryl | 6.0 |
| | Sulfate. Cocamide MEA. | |
| | Laureth-10 | |
| Sodium chloride | Sodium Chloride | 1.4 |
| Citric acid monohydrate | Citric acid | 0.1 |
| crystalline | | |
| Hydrolite ® 5 | Hydrolite ® 5 Green | 0.5 |
| Dragocid Liquid | Phenoxyethanol, Parabens | 0.5 |
| Glycolipids | | 0.5 |

TABLE F9
Anti-dandruff Shampoo (Amounts in % b.w.)

| Ingredients | Amount |
|---|---|
| Climbazole | 0.50 |
| Sodium Laureth Sulfate | 37.00 |
| Cocamidopropyl Betaine | 8.00 |
| PEG-6 Caprylic/Capric Glycerides | 2.50 |
| Laureth-2 | 2.00 |
| Water (Aqua). Glycerol. Thymus Vulgaris (Thyme). | 0.50 |
| Flower/Leaf Extract | |
| Rosmarinus Officinalis (Rosemary) Leaf Water. Water | 0.50 |
| (Aqua). Butylene Glycol. Pentylene Glycol | |
| Bisabolol | 0.10 |
| Panthenol | 0.50 |
| Polyquaternium-10 | 0.40 |
| 1,2-pentanediol (Hydrolite ® 5) | 0.50 |
| Glycolipids | 0.30 |
| Water (Aqua) | Ad 100 |

TABLE F10
Hair conditioner with Crinipan. rinse-off (Amounts in % b.w.)

| Ingredients | INCI Name | Amount |
|---|---|---|
| Lanette ® O | Cetearyl Alcohol | 4.00 |
| Dragoxat 89 | Ethylhexyl Isononanoate | 2.00 |
| Genamin ® KDM-P | Behentrimonium Chloride | 1.00 |
| SF 1550 | Phenyl Trimethicone | 0.10 |
| Neo Heliopan ® BB | Benzophenone-3 | 0.10 |
| Crinipan ® AD | Climbazole | 0.80 |
| Glycerol 99.5 P. | Glycerol | 6.00 |
| Water | Water (Aqua) | Ad 100 |
| Actipone ® Alpha Pulp | Water (Aqua). Butylene Glycol. Malic Acid. Actinidia | 0.50 |
| | Chinensis (Kiwi)Fruit Juice. Citrus. Aurantium | |
| | Dulcis (Orange). Juice. Citrus Paradisi (Grapefruit) | |
| | Juice. Pyrus Malus (Apple) Juice. Trideceth-9. | |
| | PrunusAmygdalus Dulcis (Sweet Almond) Seed Extract | |
| Extrapone ® Bamboo P | Propylene Glycol. Water (Aqua). Butylene Glycol. | 0.50 |
| | Bambusa Vulgaris Shoot Extract | |
| Sodium Hydroxide 10% | Sodium Hydroxide | 0.40 |
| solution | | |
| Colour I | Colour | 0.60 |
| Colour II | Colour | 0.30 |
| Hydrolite ® 5 | Hydrolite ® 5 Green | 0.40 |
| Glycolipids | | 0.70 |

TABLE F11
Sprayable hair conditioner with zinc pyrithrione.
leave-on (Amounts in % b.w.)

| Ingredients | INCI Name | Amount |
|---|---|---|
| Monomuls 60-35 C | Hydrogenated Palm Glycerides | 1.70 |
| Cetiol OE | Dicaprylyl Ether | 7.20 |
| Abil 100 | Dimethicone | 3.60 |
| Dehyquart F 75 | Distearoylethyl Hydroxyethylmonium. | 4.00 |
| | Methosulfate. Cetearyl Alcohol | |
| Eumulgin B1 | Ceteareth-12 | 3.50 |
| Cetiol S | Diethylhexylcyclohexane | 7.20 |
| D-Panthenol | Panthenol | 0.10 |
| Glycerol 99.5 P. | Glycerol | 1.50 |
| Water | Water (Aqua) | Ad 100 |
| Actipone ® Rosemary | Water (Aqua). Propylene. Glycol. | 0.10 |
| | Rosmarinus Officinalis. (Rosemary) | |
| | Leaf Extract | |
| Frescolat ® ML Cryst. | Menthyl Lactate | 0.50 |
| Dragosantol100 | Bisabolol | 0.10 |
| Zinc Omadine | Zinc pyrithione | 0.10 |
| Hydrolite ® 5 | Hydrolite ® 5 | 0.40 |
| Glycolipids | | 0.5 |

TABLE F12
Hair conditioner with UV protection (Amounts in % b.w.)

| Ingredients | I NCI Name | Amount |
|---|---|---|
| Renex PEG 6000 | PEG-150 | 2.50 |
| Hair Conditioner Base | Cetyl alcohol. behentrimonium chloride. Triticum | 3.00 |
| | Vulgare (Wheat) bran extract. linoleic acid | |
| PCL-Solid | Stearyl heptanoate. stearyl caprylate | 0.50 |
| Dow Corning 5200 | Laurylmethicone copolyol | 0.50 |
| Natrosol 250 HR | Hydroxyethylcellulose | 0.50 |
| Benzophenone-4 | Benzophenone-4 | 1.00 |
| Neo Heliopan AP | Disodiumphenyldibenz-imidazole tetrasulphonate | 1.00 |
| Amino methyl propanol | Amino methyl propanol | 2.00 |
| Dow Corning 949 cationic | Amodimethicone. cetrimonium chloride. trideceth-12 | 2.00 |
| emulsion | | |
| Hydrolite ® 5 | Hydrolite ® 5 | 0.80 |
| 1.2-hexanediol | 1.2-hexanediol | 0.50 |
| Glycolipids | | 0.50 |
| Water | Water (Aqua) | Ad 100 |

TABLE 13
Shower gel (Amounts in % b.w.)

| Ingredients | INCI | Amount |
|---|---|---|
| Deionized water | Water | Ad 100 |
| Plantacare PS 10 | Sodium Laureth Sulfate. Lauryl | 20.0 |
| | Glucoside | |
| Sodium chloride | Sodium Chloride | 1.4 |
| Citric acid monohydrate | Citric Acid | 1.3 |
| crystalline | | |
| Hydrolite ® 5 | Hydrolite ® 5 | 0.6 |
| SymDiol ® 68 | 1.2-hexanediol. Caprylyl glycol | 0.4 |
| Glycolipids | | 0.4 |

TABLE F14
Shaving foam (Amounts in % b.w.)

| Ingredients | Amount |
|---|---|
| Dem. Water | 77.2 |
| Triethanolamine | 4.0 |
| Edenor L2 SM (Stearinic acid. Palmitinic acid) (Cognis) | 5.3 |
| Laureth-23 | 3.0 |
| Stearylalcohol | 0.5 |
| Sodium lauryl sulfate | 3.0 |
| Extrapone Seaweed (water. propylene glycol. potassium | 1.0 |
| iodide. Fucus Vesiculosus Extract) | |
| Dragosantol (Bisabolol. Farnesol) | 0.1 |
| 1,2-pentanediol (Hydrolite ® 5) | 1.0 |
| Glycolipids | 0.3 |
| Propane. butane 4.2 Bar | 4.0 |

TABLE F15
Depilatory cream (Amounts in % b.w.)

| Ingredients | Amount |
|---|---|
| Cetearyl alcohol | 10.0 |
| Ceteareth-12 | 2.0 |
| PCL-Liquid (Cetearylethylhexanoate. Isopropylmyristate) | 3.0 |
| Dragosantol (Bisabolol. Farnesol) | 0.1 |
| Edenor L2 SM (Stearinic acid. Palmitinic acid) | 1.0 |
| Dem. Water | 52.2 |
| Urea | 5.0 |
| Dem. Water | 10.0 |
| Calcium thioglycolate | 6.0 |
| Sodium hydroxide solution. 10% | 10.0 |
| 1,2-pentanediol (Hydrolite ® 5) | 0.5 |
| Glycolipids | 0.4 |

TABLE F16
After Shave Tonic (Amounts in % b.w.)

| Ingredients | INCI | Amount |
|---|---|---|
| SymSol ® PF-3 | Water (Aqua). Pentylene Glycol. Sodium Lauryl | 3.00 |
| | Sulfoacetate. SodiumOleoyl Sarcosinate. Sodium | |
| | Chloride. Disodium Sulfoacetate. SodiumOleate. | |
| | Sodium Sulfate | |
| SymSitive ® 1609 | Pentylene Glycol. 4-t-Butylcyclohexan ol | 1.00 |
| Frescolat ® ML | Menthyl Lactate | 0.30 |
| Glycerol 99.5 P. | Glycerol | 5.00 |
| Water | Water (Aqua) | Ad 100 |
| Extrapone ® Glacier | Glycerol. Water (Aqua) | 1.00 |
| Water GW | | |
| SymCalmin ® | Butylene Glycol. Pentylene Glycol. | 0.50 |
| | Hydroxyphenyl Propamidobenzoic Acid | |
| Dragosine ® | Carnosine | 0.10 |
| Hydrolite ® 5 | Pentylene Glycol | 5.00 |
| Ethanol 96% | Alcohol Denat. | 5.00 |
| Colour Pigment | Colour Pigment | 0.05 |
| Hydrolite ® 5 | 1,2-pentanediol | 0.15 |
| Glycolipids | Hydroxyacetophenone | 1.00 |

TABLE F17
Deodorant formulation in the form of
a roll-on gel (Amounts in % b.w.)

| Ingredients | Amount |
|---|---|
| 1.3-butylene glycol | 2.00 |
| PEG-40-hydrogenated castor oil | 2.00 |
| Hydroxyethylcellulose | 0.50 |
| 1,2-pentanediol (Hydrolite ® 5) | 0.30 |
| 1.3-propanediol | 0.50 |
| 3-phenylpropanol | 0.40 |
| Ethylhexyl glycerin | 0.10 |
| Glycolipids | 0.50 |
| Water | ad 100.00 |

TABLE F18
Clear deo anti-perspirant roll-on (Amounts in % b.w.)

| Ingredients | INCI | Amount |
|---|---|---|
| Methocel E4M Premium | Hydroxypropyl Methylcellulose | 0.50 |
| Water | Water (Aqua) | Ad 100 |
| Neo-PCL Water Soluble N | Trideceth-9. PEG-5 Ethylhexanoate. Water | 1.00 |
| | (Aqua) | |
| Solubilizer | PEG-40 Hydrogenated Castor Oil. Trideceth- | 3.00 |
| | 9. Propylene Glycol. Water (Aqua) | |
| Deolite | Dimethyl Phenylpropanol. Pentylene Glycol | 0.50 |
| Locron LW | Aluminium Chlorohydrate | 25.00 |
| Aloe Vera Gel Concentrate 10/1 | Aloe Barbadensis Leaf Juice | 1.00 |
| Propylene Glycol -1.2 99 P GC | Propylene Glycol | 4.00 |
| Ethanol 96% | Alcohol Denat. | 30.00 |
| Hydrolite ® 5 | 1,2pentanediol | 1.00 |
| Glycolipids | Hydroxyacetophenone | 0.25 |

TABLE F19
Deodorant stick (Amounts in % b.w.)

| Ingredients | Amount |
|---|---|
| Sodium stearate | 8.00 |
| PPG-3 Myristyl ether | 70.00 |
| 1.2-propylene glycol | 10.00 |
| 1.1-dimethyl-3-phenylpropanol | 0.20 |
| 2-butyloctanoic acid | 0.20 |
| 1,2-pentanediol (Hydrolite ® 5) | 0.60 |
| Heptoxy-1.2-propanediol | 0.20 |
| Phenoxyethanol | 0.30 |
| Glycolipids | 0.50 |
| Water | Ad 100 |

TABLE F20
Zirconium suspensoid antiperspirant stick (Amounts in % b.w.)

| Ingredients | INCI | Amount |
|---|---|---|
| PCL Liquid 100 | Cetearyl ethylhexanonate | to 100 |
| Silicone Fluid 345 | Cyclomethicone | 10.00 |
| CRODACOL C90 | Cetyl Alcohol | 8.00 |
| SYNCROWAX HGLC | C18-36 Triglyceride | 8.00 |
| CRODAMOL PTC | Pentaerythritol Tetracaprylate/ | 5.00 |
| | Caprate | |
| SYNCROWAX HRC | Tribehenin | 4.00 |
| VOLPO N5 | Oleth-5 | 1.00 |
| Titanium Dioxide | | 1.00 |
| Rezal 36GP | Aluminium Tetrachlorohydrex GLY | 20.00 |
| Dry Flo C | Aluminium Starch Octenyl Succinate | 22.50 |
| Hydrolite ® 5 | 1,2-pentanediol | 0.60 |
| Preservative | Phenoxyethanol | 0.40 |
| Hexoxy-1.2-propanediol | | 0.10 |
| Glycolipids | | 0.40 |

TABLE F21
Deodorant pump spray with SymClariol (Amounts in % b.w.)

| Ingredients | INCI | Amount |
|---|---|---|
| SymClariol ® | Decylene Glycol | 0.50 |
| Solubilizer | PEG-40 Hydrogenated Castor Oil. Trideceth-9. | 4.00 |
| | Propylene Glycol. Water (Aqua) | |
| Neo-PCL Water Soluble N | Trideceth-9. PEG-5 Ethylhexanoate. Aqua | 1.50 |
| SymRelief ® | Bisabolol. Zingiber Officinale | 0.10 |
| | (Ginger) Root Extract | |
| Water | Water (Aqua) | Ad 100 |
| 1.2 Propylene Glycol | Propylene Glycol | 6.00 |
| Hydrolite ® 5 | 1,2-pentanediol | 0.40 |
| SymDiol ® 68 | 1.2-Hexanediol. Caprylyl Glycol | 0.20 |
| Glycolipids | | 0.40 |

TABLE F22
Antiperspirant formulations (Amounts in % b.w.)

| Ingredients | Amounts |
|---|---|
| Reach AZP-908 SUF | 24.00 |
| Cyclomethicone (Pentamer) | Ad 100 |
| Polydecene (Silkflo 364 NF) | 17.50 |
| Neo Heliopan OS (ethylhexyl salicylate) | 2.50 |
| L-Menthyl lactate (Frescolate ML) | 0.25 |
| Polyethylene | 3.00 |
| Hydrogenated castor oil | 2.00 |
| Promyristyl PM-3 | 7.00 |
| PEG-8 Distearate | 3.00 |
| Silicon dioxide (Cab-O-Sil M-5) | 1.00 |
| Stearyl alcohol | 15.00 |
| Octyldodecanol | 0.10 |
| 1,2-pentanediol (Hydrolite ® 5) | 0.80 |
| 3-Phenylpropanol | 0.40 |
| Glycolipids | 0.60 |

TABLE F23
Deodorant spray with Triclosan (Amounts in % b.w.)

| Ingredients | Amount |
|---|---|
| PEG-40-hydrogenated castor oil | 3.00 |
| Ethylhexylglycerol (Octoxyglycerol) | 0.80 |
| Ethanol | 40.00 |
| Citrate buffer | 0.50 |
| Triclosan ® (5-chloro-2-(2.4-dichlorophenoxy)phenol) | 0.25 |
| 1,2-pentanediol (Hydrolite ® 5) | 0.75 |
| Phenoxyethanol | 0.40 |
| Glycolipids | 0.40 |
| Water | Ad 100 |

TABLE F24
O/W lotion (Amounts in % b.w.)

| Ingredients | Amount |
|---|---|
| Paraffin oil | 5.00 |
| Isopropyl palmitate | 5.00 |
| Cetyl alcohol | 2.00 |
| Beeswax | 2.00 |
| Ceteareth-20 | 2.00 |
| PEG-20-glyceryl stearate | 1.50 |
| Glycerol | 3.00 |
| 1,2-pentanediol (Hydrolite ® 5) | 0.30 |
| Glycolipids | 0.60 |
| Water | ad 100.00 |

TABLE F25
Body lotion (Amounts in % b.w.)

| Ingredients | Amount |
|---|---|
| Cetearyl Alcohol | 2.00 |
| Ethylhexyl Isononanoate | 5.00 |
| Cetearyl Ethylhexanoate. Isopropyl Myristate | 3.00 |
| Glyceryl Oleate Citrate. Caprylic/Capric Triglyceride | 4.00 |
| Water (Aqua) | 79.50 |
| Carbomer | 0.30 |
| Sodium Benzoate | 0.100 |
| Propylene Glycol | 5.00 |
| Sodium Hydroxide 30% solution | 0.30 |
| 1,2-pentanediol (Hydrolite ® 5) | 0.30 |
| Glycolipids | 0.20 |

TABLE F26
Cream (Amounts in % b.w.)

| Ingredients | Amount |
|---|---|
| Paraffin oil | 10.00 |
| Ozokerite | 4.00 |
| Vaseline | 4.00 |
| Vegetable oil | 10.00 |
| Wool wax alcohol | 2.00 |
| Aluminum stearate | 0.40 |
| 1,2-pentanediol (Hydrolite ® 5) | 2.00 |
| Phenoxyethanol | 0.50 |
| Glycolipids | 0.50 |
| Water | ad 100.00 |

TABLE F27
Cream (Amounts in % b.w.)

| Ingredients | INCI | Amount |
|---|---|---|
| Dracorin ® CE | Glyceryl Stearate Citrate | 1.00 |
| Lanette ® O | Cetearyl Alcohol | 2.00 |
| Cutina ® GMS-V | Glyceryl Stearate | 1.00 |
| Tegosoft ® MM | Myristyl Myristate | 1.00 |
| Xiameter ® PMX-0246. Cyclosiloxane | Cyclohexasiloxane (and) Cyclopentasiloxane | 0.50 |
| Dragoxat ® 89 | Ethylhexyl Isononanoate | 2.00 |
| PCL-Liquid 100 | Cetearyl Ethylhexanoate | 4.00 |
| Neutral Oil | Caprylic/Capric Triglyceride | 4.00 |
| Carbopol ® Ultrez 21 | Acrylates/C10-30 Alkyl Acrylate Crosspolymer | 0.20 |
| Keltrol ® CG-T | Xanthan Gum | 0.10 |
| Water | Water (Aqua) | Ad 100 |
| Glycerol 99.5 P. | Glycerol | 3.00 |
| Propylene Glycol -1.2 99 P GC | Propylene Glycol | 2.00 |
| Sodium Benzoate | Sodium Benzoate | 0.10 |
| Sodium Hydroxide 10% solution | Sodium Hydroxide | 0.50 |
| Hydrolite ® 5 | 1,2-pentanediol | 0.30 |
| Glycolipids | | 0.70 |

TABLE F28
Hand and body cream (Amounts in % b.w.)

| Ingredients | INCI | Amount |
|---|---|---|
| Dracorin ® GOC | Glyceryl Oleate Citrate. Caprylic/Capric Triglyceride | 2.00 |
| PCL-Solid | Stearyl Heptanoate. Stearyl Caprylate | 2.50 |
| Lanette ® O | Cetearyl Alcohol | 1.50 |
| Cutina ® GMS-V | Glyceryl Stearate | 1.00 |
| Dragoxat ® 89 | Ethylhexyl Isononanoate | 3.00 |
| PCL-Liquid 100 | Cetearyl Ethylhexanoate | 7.00 |
| Isodragol ® | Triisononanoin | 4.00 |
| Xiameter ® PMX-0345 Cyclosiloxane | Cyclopentasiloxane (and) Cyclohexasiloxane | 0.50 |
| Water | Water (Aqua) | Ad 100 |
| Carbopol ® Ultrez 21 | Acrylates/C10-30 Alkyl Acrylate Crosspolymer | 0.20 |
| Keltrol ® CG-RD | Xanthan Gum | 0.10 |
| Glycerol 85 P. | Glycerol | 3.00 |
| DragoBetaGlucan | Water (Aqua). Butylene Glycol. Glycerol. Avena | 1.50 |
| | Sativa (Oat) Kernel Extract | |
| Potassium Sorbat | Potassium Sorbate | 0.10 |
| Sodium Hydroxide 10% solution | Sodium Hydroxide | 0.50 |
| Hydrolite ® 5 | 1,2-pentanediol | 0.20 |
| Euxyl ® K300 | Methyl-. Butyl-. Ethyl-. Propyl. Isobutylparaben. | 0.30 |
| | Phenoxyethanol. | |
| Glycolipids | | 0.30 |

TABLE F29
Face cream (Amounts in % b.w.)

| Ingredients | INCI | Amount |
|---|---|---|
| Emulsiphos ® | Potassium Cetyl Phosphate. Hydrogenated Palm | 1.50 |
| | Glycerides | |
| Cutina ® GMS-V | Glyceryl Stearate | 1.70 |
| Lanette ® O | Cetearyl Alcohol | 3.00 |
| Tegosoft ® MM | Myristyl Myristate | 1.00 |
| PCL-Liquid 100 | Cetearyl Ethylhexanoate | 1.00 |
| Isodragol ® | Triisononanoin | 3.00 |
| Dragoxat ® 89 | Ethylhexyl Isononanoate | 4.00 |
| Avocado Oil | Persea Gratissima (Avocado) Oil | 3.00 |
| Abil ® 350 | Dimethicone | 0.50 |
| Covi-ox ® T-70 | Tocopherol | 0.10 |
| Edeta ® BD | Disodium EDTA | 0.10 |
| Carbopol ® Ultrez 21 | Acrylates/C10-30 Alkyl Acrylate Crosspolymer | 0.30 |
| Keltrol ® CG-RD | Xanthan Gum | 0.150 |
| Water | Water (Aqua) | Ad 100 |
| Glycerol 99.5 P. | Glycerol | 4.00 |
| Propylene Glycol -1.2 99 P GC | Propylene Glycol | 3.00 |
| SymMatrix ® | Maltodextrin. Rubus Fruticosus (Blackberry) Leaf | 0.50 |
| | Extract | |
| Sodium Hydroxide 10% solution | Sodium Hydroxide | 0.50 |
| Hydrolite ® 5 | 1,2-pentanediol | 0.30 |
| Euxyl ® K712 | Sodium Benzoate. Potassium Sorbate | 0.20 |
| Glycolipids | | 0.60 |

TABLE F30
Moisturizing body care cream (Amounts in % b.w.)

| Ingredient | Amount |
|---|---|
| PEG-7 hydrogenated castor oil | 6.00 |
| Cetearyl ethyl hexanoate | 10.00 |
| Isopropyl myristate | 5.00 |
| Mineral oil | 7.00 |
| Shea Butter (Butyrospermum parkii) | 0.50 |
| Aluminum stearate | 0.50 |
| Magnesium stearate | 0.50 |
| Bisabolol | 0.20 |
| Quaternium-18-Hectorit | 0.70 |
| Dipropylene glycol | 5.0 |
| Magnesium sulfate | 0.70 |
| 1,2-pentanediol (Hydrolite ® 5) | 4.00 |
| Glycolipids | 0.40 |
| Aqua dem. | 58.90 |

TABLE F31
Anti-wrinkle cream (Amounts in % b.w.)

| Ingredients | Amount |
|---|---|
| Glyceryl Stearate Citrate | 1.00 |
| Glyceryl Laurate | 1.00 |
| Cetearyl Alcohol (and) Myristyl Myristate | 3.00 |
| Cetearyl Ethylhexanoate | 4.00 |
| Mineral oil | 4.00 |
| Cyclohexasiloxane | 0.50 |
| Acrylates/C10-30 Alkyl Acrylate Crosspolymer | 0.20 |
| Water | Ad 100 |
| 1.2-Hexanediol | 2.00 |
| Sodium Hydroxide 10% solution | 0.10 |
| Narcissus Tazetta Bulb Extract | 1.00 |
| 1,2-pentanediol (Hydrolite ® 5) | 0.30 |
| Glycolipids | 0.50 |

TABLE F32
Functional skin oil for disinfection (Amounts in % b.w.)

| Ingredients | INCI | Amount |
|---|---|---|
| Neutral Oil | Caprylic/Capric Triglyceride | Ad 100 |
| Sweet Almond Oil | Prunus Dulcis | 20.00 |
| Dragoxat ® 89 | Ethylhexyl Isononanoate | 4.00 |
| Isopropyl Palmitate | Isopropyl Palmitate | 6.00 |
| PCL-Liquid 100 | Cetearyl Ethylhexanoate | 15.00 |
| Dragosantol ® 100 | Bisabolol | 0.20 |
| Retinyl Acetate In Oil | Retinyl Acetate | 0.50 |
| (1 Mio. le/G) | | |
| Vitamin E Acetate | Tocopheryl Acetate | 0.50 |
| Covi-ox ® T-70 | Tocopherol | 0.10 |
| Hydrolite ® 5 | 1,2-pentanediol | 0.30 |
| Preservative | Methyl-. Butyl-. Ethyl-. | 0.30 |
| | Propylparaben | |
| Glycolipids | | 0.40 |

TABLE F33
Septic wound cream (Amounts in % b.w.)

| Ingredients (INCI) | Amount |
|---|---|
| Sorbitan Isostearate. Hydrogenated Castor Oil. Ceresin. | 6.00 |
| Beeswax (Cera Alba) | |
| Petrolatum | 21.00 |
| Cera Alba | 5.00 |
| Cetearyl Alcohol | 7.00 |
| Prunus Dulcis | 7.00 |
| Lanolin | 5.00 |
| Paraffinum Liquidum | 12.00 |
| Water (Aqua) | Ad 100 |
| Panthenol | 7.00 |
| Magnesium Sulfate | 0.70 |
| 1,2-pentanediol (Hydrolite ® 5) | 1.00 |
| Tocopheryl Acetate | 1.00 |
| Phenoxyethanol | 0.50 |
| Glycolipids | 0.50 |

TABLE F34
Moisturizing and disinfecting face mask (Amounts in % b.w.)

| Ingredients | INCI | Amount |
|---|---|---|
| Water | Water (Aqua) | Ad 100 |
| Stabileze QM | PVM/Ma Decadiene Crosspolymer | 0.50 |
| Biotive ® L-Arginine | Arginine | 0.75 |
| Actipone ® Laminaria Saccharina GW | Glycerol. Water (Aqua). Laminaria Saccharina | 1.00 |
| | Extract | |
| Extrapone ® Cucumber | Water (Aqua). Propylene Glycol. Cucumis Sativus | 1.00 |
| | (Cucumber) Juice | |
| Glycerol 99.5 P. | Glycerol | 7.00 |
| Neo Actipone ® Soap Nutshell | Sapindus Mukurossi Peel Extract | 0.50 |
| Colour I | Colour | 0.01 |
| Hydrolite ® 5 | Pentylene Glycol | 5.00 |
| Solubilizer | PEG-40 Hydrogenated Castor Oil. Trideceth-9. | 0.60 |
| | Water (Aqua) | |
| Hydrolite ® 5 | 1,2-pentanediol | 0.08 |
| Glycolipids | | 0.40 |

TABLE F35
Sprayable disinfecting gel (Amounts in % b.w.)

| Ingredients | INCI | Amount |
|---|---|---|
| Water | Water (Aqua) | Ad 100 |
| Stabileze QM | PVM/Ma Decadiene Crosspolymer | 0.25 |
| Sodium Hydroxide 10% solution | Sodium Hydroxide | 0.40 |
| Coffein pure | Caffeine | 0.50 |
| Extrapone ® Horse Chestnut | Propylene Glycol. Water (Aqua). Glucose. Aesculus | 1.00 |
| | Hippocastanum (Horse Chestnut) Seed | |
| | Extract. Lactic Acid | |
| Hydrolite ® 5 | 1,2-pentanediol | 3.00 |
| 1.3 Butylene Glycol | Butylene Glycol | 5.00 |
| Biotive ® Esculin Sesquihydrate | Esculin | 0.30 |
| Ethanol 96% | Alcohol Denat. | 10.00 |
| Solubilizer | PEG-40 Hydrogenated Castor Oil. Trideceth-9. | 0.50 |
| | Water (Aqua) | |
| Glycolipids | | 0.30 |

TABLE F36
Mineral wash and cleaning gel (Amounts in % b.w.)

| Ingredients | INCI | Amount |
|---|---|---|
| Water | Water (Aqua) | Ad 100 |
| Pionier ® NP 37 G | Sodium Carbomer | 1.50 |
| SymSol ® PF-3 | Water (Aqua). Pentylene Glycol. Sodium Lauryl | 5.00 |
| | Sulfoacetate. SodiumOleoyl Sarcosinate. Sodium | |
| | Chloride. Disodium Sulfoacetate. SodiumOleate. | |
| | Sodium Sulfate | |
| Hydroviton ® 24 | Water (Aqua). Pentylene Glycol. Glycerol. Sodium | 1.00 |
| | Lactate. Lactic Acid. Serine. Urea. Sorbitol. | |
| | Sodium Chloride. Allantoin | |
| Extrapone ® Silk GW | Water (Aqua). Glycerol. Hydrolyzed Silk | 1.00 |
| Hydrolite ® 5 Green | Hydrolite ® 5 Green | 4.00 |
| Actipearls Red Star # DH10402/6 | Water (Aqua). Propylene Glycol. Algin. Gellan | 1.00 |
| | Gum. Xanthan Gum. Calcium Chloride. CI | |
| | 12490 (Pigment Red 5). Titanium Dioxide (CI | |
| | 77891) | |
| 1,2-pentanediol (Hydrolite ® 5) | 1,2-pentanediol | 0.50 |
| 3-Phenylpropanol | | 0.70 |
| Glycolipids | | 0.30 |

TABLE F37
Anti-acne wash (Amounts in % b.w.)

| Ingredients (INCI) | Amount |
|---|---|
| Water (Aqua) | 45.70 |
| Polyquaternium-7 | 0.50 |
| Cocamidopropyl Betaine 9.000 | 9.00 |
| Coco Glucoside 2.000 | 2.00 |
| Polysorbate 80. Glycerol. Gossypium Herbaceum. (Cotton) | 1.00 |
| Seed Oil. Water (Aqua) | |
| Trideceth-9. PEG-5 Ethylhexanoate. Water (Aqua) | 1.00 |
| Glycereth-90 Isostearate. Laureth-2 | 0.50 |
| Sodium Laureth Sulfate 37.000 | 37.00 |
| Glycerol. Triticum Vulgare (Wheat) Gluten. Water (Aqua) | 1.00 |
| Sodium Chloride | 0.30 |
| 1,2-pentanediol (Hydrolite ® 5) | 1.00 |
| Glycolipids | 0.50 |

TABLE F38
Cosmetic sun protection composition (Amounts in % b.w.)

| Ingredient | Amount |
|---|---|
| Ethylhexyl cinnamic acid | 7.50 |
| Benzophenon-3 | 2.00 |
| Polyglyceryl dimer soyate | 0.80 |
| Sorbitane stearate | 1.00 |
| Tocopheryl acetate | 0.50 |
| Glyceryl stearate. PEG-100 Stearate | 3.00 |
| PEG-40. hydrogenated castor oil | 1.00 |
| Titanium dioxide. aluminum oxide hydrate. | 3.00 |
| Dimethicon/Methicon Copolymer | |
| Butyrospermum parkii (Shea Butter) | 1.00 |
| C₁₂₋₁₅ alkyl benzoate | 6.50 |
| Butylene glycol | 5.00 |
| Xanthan gum | 0.30 |
| Disodium EDTA | 0.10 |
| Allantoin | 0.10 |
| Polyacryl amide. C₁₃₋₁₄ isoparaffin. Laureth-7 | 1.00 |
| 1,2-pentanediol (Hydrolite ® 5) | 5.00 |
| 4-t Butylcyclohexanol | 1.00 |
| Glycolipids | 0.60 |
| Aqua dem. | Ad 100 |

TABLE F39
Sun protection spray (Amounts in % b.w.)

| Ingredients | INCI | Amount |
|---|---|---|
| Water. demineralized | Water (aqua) | 69.50 |
| Glycerol | Glycerol | 4.00 |
| 1.3 butylene glycol | Butylene glycol | 5.00 |
| D-Panthenol | Panthenol | 0.50 |
| Lara Care A-200 | Galactoarabinan | 0.25 |
| Baysilone oil M 10 | Dimethicone | 1.00 |
| Edeta BD | Disodium EDTA | 0.10 |
| Copherol 1250 | Tocopheryl acetate | 0.50 |
| Cetiol OE | Dicaprylyl ether | 3.00 |
| Neo Heliopan ® HMS | Homosalate | 5.00 |
| Neo Heliopan ® AV | Ethylhexyl methoxycinnamate | 6.00 |
| Neo Heliopan ® 357 | Butyl methoxydibenzoylmethane | 1.00 |
| Corapan TQ | Diethylhexylnaphthalate | 2.00 |
| Alpha Bisabolol | Bisabolol | 0.10 |
| Pemulen TR-2 | Acrylates/C10-30 alkyl | 0.25 |
| | acrylate crosspolymer | |
| NaOH. 10% | Sodium hydroxide | 0.60 |
| Hydrolite ® 5 | 1,2-pentanediol | 0.20 |
| Glycolipids | | 0.50 |

TABLE F40
Sunscreen spray O/W. SPE 15-20 (Amounts in % b.w.)

| Ingredients | INCI | Amount |
|---|---|---|
| Dracorin ® GOC | Glyceryl Oleate Citrate. Caprylic/Capric | 2.00 |
| | Triglyceride | |
| Corapan ® TQ | Diethylhexyl 2.6-Naphthalate | 3.00 |
| Neo Heliopan ® HMS | Homosalate | 7.00 |
| Neo Heliopan ® OS | Ethylhexyl Salicylate | 5.00 |
| Neo Heliopan ® 357 | Butyl Methoxydibenzoylmethane | 3.00 |
| Isoadipate | Diisopropyl Adipate | 6.00 |
| Baysilone ® Oil M10 | Dimethicone | 1.00 |
| Edeta ® BD | Disodium EDTA | 0.10 |
| Vitamin E Acetate | Tocopheryl Acetate | 0.50 |
| Dragosantol ® 100 | Bisabolol | 0.10 |
| Pemulen ® TR-2 | Acrylates/C10-30 Alkyl Acrylate Crosspolymer | 0.25 |
| Water | Water (Aqua) | Ad 100 |
| Glycerol 99.5 P. | Glycerol | 4.00 |
| Butylene Glycol | Butylene Glycol | 5.00 |
| Neo Heliopan ® Hydro (103089). | Phenylbenzimidazole Sulfonic Acid | 8.00 |
| used as 25% aq. solution | | |
| Biotive ® L-Arginine | Arginine | 0.55 |
| Hydrolite ® 5 | 1,2-pentanediol | 0.40 |
| Sobrol M | Methylparaben | 0.30 |
| Glycolipids | | 0.60 |

TABLE F41
Sun protection soft cream (W/O). SPF 40 (Amounts in % b.w.)

| Ingredients | INCI | Amount |
|---|---|---|
| Dehymuls PGPH | Polyglyceryl-2 | 5.00 |
| | dipolyhydroxystearate | |
| Copherol 1250 | Tocopheryl acetate | 0.50 |
| Permulgin 3220 | Ozocerite | 0.50 |
| Zinc stearate | Zinc stearate | 0.50 |
| Tegosoft TN | C12-15 Alkyl benzoate | 10.00 |
| Neo Heliopan ® E1000 | Isoamyl-p-methoxycinnamate | 2.00 |
| Neo Heliopan ® 303 | Octocrylene | 5.00 |
| Neo Heliopan ® MBC | 4-Methylbenzylidene camphor | 3.00 |
| Zinc oxide. neutral | Zinc oxide | 5.00 |
| Water. distilled | Water (aqua) | Add 100 |
| EDETA BD | Disodium EDTA | 0.10 |
| Glycerol | Glycerol | 4.00 |
| Magnesium sulfate | Magnesium sulfate | 0.50 |
| Hydrolite ® 5 | 1,2-pentanediol | 0.30 |
| Symdiol ® 68 | 1.2-Hexanediol. Caprylylglycol | 0.30 |
| Glycolipids | | 0.80 |

TABLE F42
Sun protection milk (W/O) (Amounts in % b.w.)

| Ingredients | INCI | Amount |
|---|---|---|
| Dehymuls PGPH | Polyglyceryl-2 dipolyhydroxystearate | 3.00 |
| Beeswax 8100 | Beeswax | 1.00 |
| Monomuls 90-0-18 | Glyceryl oleate | 1.00 |
| Zinc stearate | Zinc stearate | 1.00 |
| Cetiol SN | Cetearyl isononanoate | 5.00 |
| Cetiol OE | Dicaprylyl ether | 5.00 |
| Tegosoft TN | C12-15 alkyl benzoate | 4.00 |
| Vitamin E | Tocopherol | 0.50 |
| Neo Heliopan ® OS | Ethylhexyl salicylate | 5.00 |
| Neo Heliopan ® AV | Ethylhexyl methoxycinnamate | 7.50 |
| Uvinul ® T150 | Ethylhexyl triazone | 1.50 |
| Water. distilled | Water (Aqua) | To 100 |
| Trilon BD | Disodium EDTA | 0.10 |
| Glycerol | Glycerol | 5.00 |
| Neo Heliopan ® AP 10% solution. | Disodium phenyl dibenzimidazole | 15.00 |
| neutralized with NaOH | tetrasulfonate | |
| Hydrolite ® 5 | 1,2-pentanediol | 0.25 |
| Alpha bisabolol | Bisabolol | 0.10 |
| SymOcide ® PT | Phenoxyethanol. Tropolone | 0.25 |
| Glycolipids | | 0.25 |

TABLE F43
After sun gel (Amounts in % b.w.)

| Ingredients | INCI | Amount |
|---|---|---|
| SymSol ® PF-3 | Water (Aqua). Pentylene Glycol. Sodium Lauryl | 3.000 |
| | Sulfoacetate. SodiumOleoyl Sarcosinate. | |
| | Sodium Chloride. Disodium Sulfoacetate. | |
| | SodiumOleate. Sodium Sulfate | |
| Glycerol 99.5 P. | Glycerol | 5.000 |
| SymHelios ® 1031 | Benzylidene Dimethoxydimethylin danone | 0.100 |
| Water | Water (Aqua) | Ad 100 |
| Pemulen ® TR-2 | Acrylates/C10-30 Alkyl Acrylate Crosspolymer | 1.000 |
| D-Panthenol 75 W | Panthenol | 0.500 |
| SymFinity ® 1298 | Echinacea Purpurea Extract | 0.100 |
| Extrapone ® Pearl GW | Water (Aqua). Glycerol. Hydrolyzed Pearl. | 1.000 |
| | Xanthan Gum | |
| Sodium Hydroxide 10% solution | Sodium Hydroxide | 2.500 |
| Ethanol 96% | Alcohol Denat. | 15.000 |
| Hydrolite ® 5 | 1,2-pentanediol | 0.20 |
| Glycolipids | | 0.50 |

TABLE F44
After sun lotion (Amounts in % b.w.)

| Ingredients | Amount |
|---|---|
| Acrylate/C10-30 alkylacrylate crosspolymer | 0.4 |
| Cetearylethyl hexanoate | 15.0 |
| Bisabolol | 0.2 |
| Tocopheryl acetate | 1.0 |
| Panthenol | 1.0 |
| Alcohol | 15.0 |
| Glycerol | 3.0 |
| 1.2-Hexanediol | 0.60 |
| Glycolipids | 0.30 |
| 1,2-pentanediol (Hydrolite ® 5) | 4.0 |
| Aqua dem. | Ad 100 |
| Triethanolamine | 0.2 |

TABLE F45
Hair styling gel (Amounts in % b.w.)

| Ingredients | Amount |
|---|---|
| Water | Ad 100 |
| PVM/MA Decadiene Crosspolymer | 0.60 |
| PVP | 3.00 |
| Isocetyl Stearate | 4.00 |
| Ethylhexyl Methoxycinnamate | 0.50 |
| Aminomethyl Propanol | 0.40 |
| 1,2-pentanediol (Hydrolite ® 5) | 0.60 |
| SymDiol ® 68T (1.2-Hexanediol. 1.2-Octanediol. Tropolone) | 0.30 |
| Phenoxyethanol | 0.20 |
| Glycolipids | 0.40 |

TABLE F46
Silicone emulsion (Amounts in % b.w.)

| Ingredients | Amount |
|---|---|
| Potassium Cetyl Phosphate. Hydrogenated | 1.00 |
| Palm Glycerides | |
| Cyclohexasiloxane | 4.00 |
| Cetearyl Alcohol | 1.50 |
| Phenyl Trimethicone | 3.00 |
| Stearyl Heptanoate. Stearyl Caprylate | 3.00 |
| Dimethicone | 1.00 |
| Xanthan Gum | 0.20 |
| Isoamyl p-Methoxycinnamate | 5.00 |
| Water | Ad 100 |
| Methylpropanediol | 3.00 |
| 1,2-pentanediol (Hydrolite ® 5) | 0.30 |
| Diazolidinyl urea | 0.10 |
| Glycolipids | 0.50 |

TABLE F47
Microemulsion gel (Amounts in % b.w.)

| Ingredient | Amount |
|---|---|
| Glycerol isostearate | 1.80 |
| Octoxyglycerol | 1.00 |
| Ceteareth-15 | 5.20 |
| PEG-150 Distearate | 1.00 |
| Aluminium chlorohydrate | 5.00 |
| Isotridecylisononanoate | 3.30 |
| Cyclomethicone | 6.60 |
| 1,2-pentanediol (Hydrolite ® 5) | 0.70 |
| Euxyl ® K145 (Methylchloroisothiazolinone. | 0.10 |
| Methylisothiazlinone. Bronopol) | |
| Glycolipids | 0.40 |
| Water | Ad 100 |

TABLE F48
Air freshener in gel form (Amounts in % b.w.)

| Ingredient | Amount |
|---|---|
| Demineralised water | Ad 100 |
| Genugel ® X-6424 (carrageenan) | 2.00 |
| Arkopal ® N 100 or Tergitol ® NP 10 (Emulsifer) | 3.50 |
| 1,2-pentanediol (Hydrolite ® 5) | 0.60 |
| Preventol ® D 7 (5-chloro-2-methyl-4-isothiazolin-3-one. | 0.10 |
| 2-methyl-2H isothiazol-3-one) | |
| Glycolipids | 0.80 |

TABLE F49
Cleaner. APC liquid. alkaline pH 8-10 (Amounts in % b.w.)

| Ingredients (INCI) | Amount |
|---|---|
| Aqua | 59.06 |
| Tri Sodium Citrate Dihydrate | 3.00 |
| Sodium Laureth Sulfate | 30.00 |
| Trideceth-9 | 5.00 |
| Ethanol | 2.00 |
| Citric Acid 10% solution | 0.24 |
| 1,2-pentanediol (Hydrolite ® 5) | 0.50 |
| Mixture of 5-Chloro-2-methyl-2H-isothiazol-3-one | 0.10 |
| and 2-Methyl-2H-isothiazol-3-one | |
| Glycolipids | 0.30 |

TABLE F50
Fabric softener (Amounts in % b.w.)

| Ingredients (INCI) | Amount |
|---|---|
| Aqua | 72.10 |
| Dialkylester ammomium methosulfate | 16.60 |
| Polydimethylsiloxane | 0.30 |
| Magnesiumchloride | 10.00 |
| 1,2-pentanediol (Hydrolite ® 5) | 0.60 |
| Mixture of 5-Chloro-2-methyl-2H-isothiazol-3-one | 0.10 |
| and 2-Methyl-2H-isothiazol-3-one | |
| Glycolipids | 0.40 |

TABLE F51
Liquid detergent (Amounts in % b.w.)

| Ingredients | Amount |
|---|---|
| Deionized water | 39.60 |
| Optical brightener | 0.10 |
| Coconut fatty acids (C12-C18) | 7.50 |
| Potassium hydroxide 50% solution | 4.30 |
| Propane-1.2-diol | 5.00 |
| Fatty alcohols C12-C15. 8 EO | 12.00 |
| Na-salt of secondary alkyl | 17.00 |
| sulfonates (C13-C17) | |
| Triethanolamine | 2.00 |
| Trisodium citrate dihydrate | 5.00 |
| Dequest 2066 Diethylenetriamine | 3.00 |
| penta(methylene phosphonic acid) | |
| Ethanol | 3.00 |
| Enzymes | 0.70 |
| 1,2-pentanediol (Hydrolite ® 5) | 0.50 |
| Glycolipids | 1.00 |

TABLE F52
Liquid detergent concentrate (Amounts in % b.w.)

| Ingredients | Amount |
|---|---|
| Deionized water | 12.9 |
| Coconut fatty acids (C12-C18) | 10.0 |
| Fatty alcohols C12-C15. 8 EO | 26.0 |
| Na-salt of secondary alkyl sulfonates (C13-C17) | 26.5 |
| Triethanol amine | 8.5 |
| Na-salt of fatty alcohol sulfates C12-C14 | 3.0 |
| Ethanol | 5.5 |
| Urea | 4.5 |
| Enzymes | 0.9 |
| Citric acid | 1.0 |
| 1,2-pentanediol (Hydrolite ® 5) | 0.7 |
| Glycolipids | 0.8 |

TABLE F53
Toilet cleaner (Amounts in % b.w.)

| Ingredients | Amount |
|---|---|
| Water | 93.0 |
| Kelzan ASX-T | 0.5 |
| Parafin sulfonate. sodium salt | 1.0 |
| Citric acid | 5.0 |
| Colorant (FD & C Yellow No. 6) | 0.1 |
| 1,2-pentanediol (Hydrolite ® 5) | 0.3 |
| Preservative (Benzisothiazolinone. Glutaral) | 0.05 |
| Glycolipids | 0.6 |

TABLE F54
Dish washing concentrate (Amounts in % b.w.)

| Ingredients | Amount |
|---|---|
| Sodium laurylsulfate | 31.0 |
| Propane-1.2-diole | 6.0 |
| Ethyl alcohol 96% | 7.0 |
| Palm tree glucosides | 6.0 |
| Coco betaine | 18.0 |
| 1,2-pentanediol (Hydrolite ® 5) | 0.4 |
| Glycolipids | 0.5 |
| Water | 31.6 |

TABLE F55
Dish washing concentrate (Amounts in % b.w.)

| Ingredients | Amount |
|---|---|
| Palm tree glucosides | 4.0 |
| Sodium lauryl sulfate | 45.0 |
| Coco betaine | 8.0 |
| Ethyl alcohol 96% | 1.0 |
| Colorant (C.I. Pigment Blue 15) | 0.05 |
| 1,2-pentanediol (Hydrolite ® 5) | 0.2 |
| Glycolipids | 0.7 |
| Water | Ad 100 |

TABLE F56
Solution for wet wipes (Amounts in % b.w.)

| Ingredients | INCI | Amount |
|---|---|---|
| SymSol ® PF-3 | Water (Aqua). Pentylene Glycol. Sodium Lauryl | 2.00 |
| | Sulfoacetate. SodiumOleoyl Sarcosinate. | |
| | Sodium Chloride. Disodium Sulfoacetate. | |
| | SodiumOleate. Sodium Sulfate | |
| Dragosantol ® 100 | Bisabolol | 0.10 |
| Glycerol 99.5 P. | Glycerol | 5.00 |
| Water | Water (Aqua) | Ad 100 |
| Hydrolite ® 5 | 1,2-pentanediol | 5.00 |
| D-Panthenol 75 W | Panthenol | 0.80 |
| DragoCalm ® | Water (Aqua). Glycerol. Avena Sativa (Oat) | 1.00 |
| | Kernel Extract | |
| Witch Hazel-Distillate | Hamamelis Virginiana (Witch Hazel) Water. | 1.00 |
| | Water (Aqua). Alcohol | |
| Allplant Essence ® Org. Rose | Pelargonium Graveolens Flower/Leaf/Stem | 1.00 |
| Geranium P | Water | |
| Glycolipids | | 0.50 |

What claimed is:

1. A method for improving antimicrobial stability of a composition, against at least one of the following micro-organisms
Asaia siamensis
Bacillus licheniformis
Bacillus subtilis
Byssochlamys fulva
Byssochlamys nivea
Candida parapsilosis
Clostridium perfringens
Clostridium sporogenes
Corynebacterium xerosis
Enterococcus faecalis
Lactobacillus paracasei ssp. paracasei
Leuconostoc lactis
Leuconostoc mesenteroides
Malassezia furfur
Neosartorya fischeri
Penicillium paneum
Saccharomyces cerevisiae
Yarrowia lipolytica
Zygosaccharomyces bailii
comprising or consisting of the following steps:
(a) providing a composition in need of such improved antimicrobial stability;
(b) providing at least one glycolipid and optionally at least one other antimicrobial agent;
(c) adding a working amount of said at least one glycolipid and optionally said at least one other anti-microbial agent to said composition;
(d) optionally providing at least one other compound to said composition affecting solubility of compounds added in step (b) and (c);
wherein said glycolipid represents an ustilagic acid esterified with a linear C₁-C₄ aliphatic alcohol.

2. The method of claim 1, wherein said composition is a cosmetic, detergent or food preparation.

3. The method of claim 1, wherein said glycolipids follow formula (I) 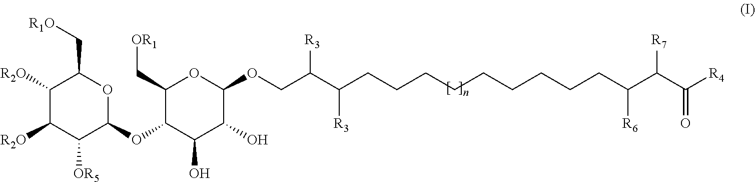
wherein independently from each other
n stands for 1 or 2,
R₁ represents hydrogen or —COCH₃;
R₂ represents H or 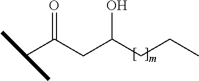
m=1, 2 or 3;
R₃ represents hydrogen or —OH;
R₄ represents —OH or —OCH3;
R₅ represents hydrogen or 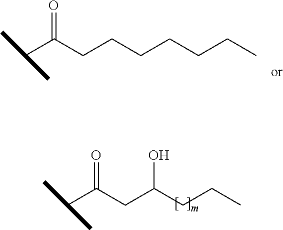
or m=1, 2 or 3;
R₆ represents hydrogen or —OH;
R₇ represents hydrogen or —OH or ═O;
or a salt of said glycolipid.

4. The method of claim 1, wherein said glycolipids follow formula (II) 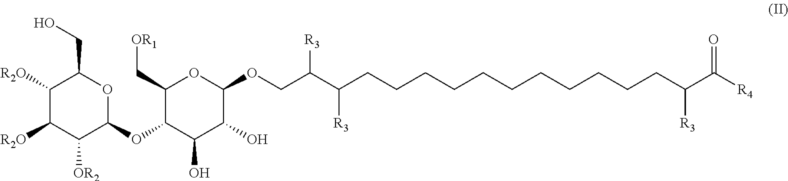
wherein independently from each other
R₁ represents hydrogen or —COCH₃;
R₂ represents H or 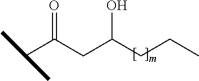
m=1, 2 or 3;
R₃ represents hydrogen or —OH;
R₄ represents —OH or —OCH3;
or a salt of said glycolipid.

5. The method of claim 1, wherein said glycolipids follow formula (III) 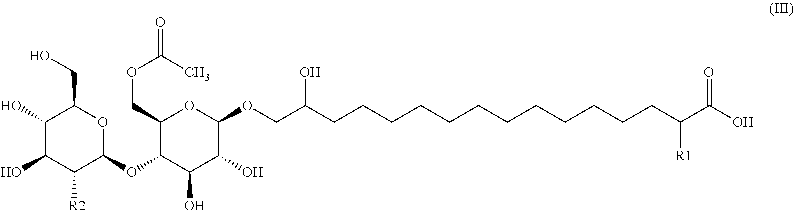
wherein independently from each other
R1 represents hydrogen or —OH;
R2 represents hydrogen or 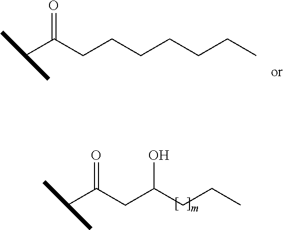
or m=1, 2 or 3;
or a salt of said glycolipid.

6. The method of claim 5, wherein said glycolipids follow at least one the following formulae (1) to (6)

| Compound | Structure |
|---|---|
| 1 | 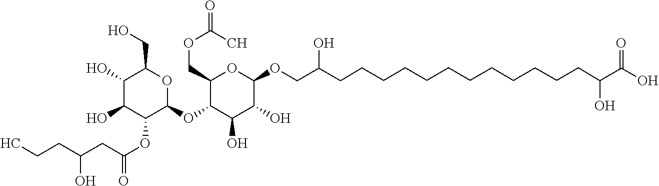 [SMILES: CCCC(O)CC(=O)O[C@H]1[C@H](O[C@H]2[C@H](O)[C@@H](O)[C@H](OCC(O)CCCCCCCCCCCCC(O)C(=O)O)O[C@@H]2COC(C)=O)O[C@H](CO)[C@@H](O)[C@@H]1O] |
| 2 | 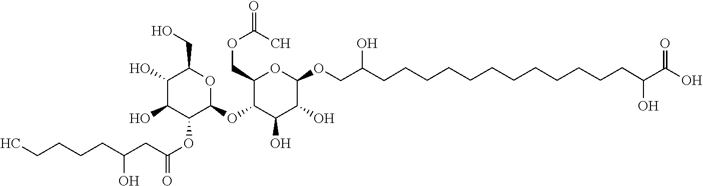 [SMILES: CCCCCC(O)CC(=O)O[C@H]1[C@H](O[C@H]2[C@H](O)[C@@H](O)[C@H](OCC(O)CCCCCCCCCCCCC(O)C(=O)O)O[C@@H]2COC(C)=O)O[C@H](CO)[C@@H](O)[C@@H]1O] |
| 3 | 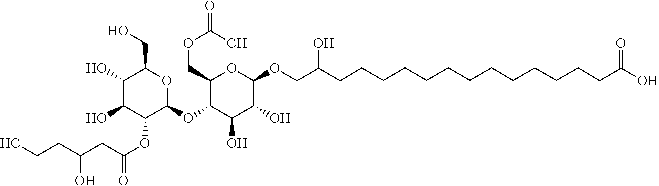 [SMILES: CCCC(O)CC(=O)O[C@H]1[C@H](O[C@H]2[C@H](O)[C@@H](O)[C@H](OCC(O)CCCCCCCCCCCCCC(=O)O)O[C@@H]2COC(C)=O)O[C@H](CO)[C@@H](O)[C@@H]1O] |
| 4 | 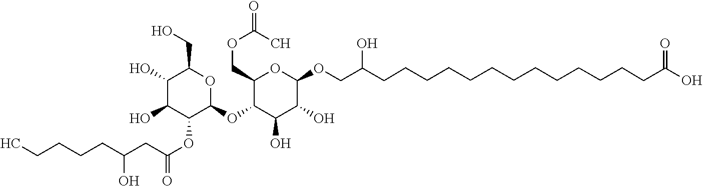 [SMILES: CCCCCC(O)CC(=O)O[C@H]1[C@H](O[C@H]2[C@H](O)[C@@H](O)[C@H](OCC(O)CCCCCCCCCCCCCC(=O)O)O[C@@H]2COC(C)=O)O[C@H](CO)[C@@H](O)[C@@H]1O] |
| 5 | 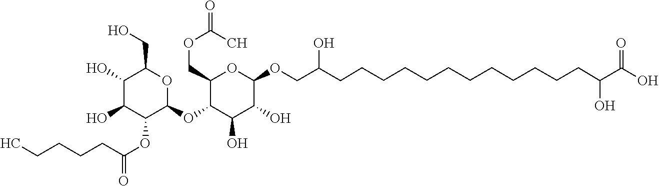 [SMILES: CCCCCC(=O)O[C@H]1[C@H](O[C@H]2[C@H](O)[C@@H](O)[C@H](OCC(O)CCCCCCCCCCCCC(O)C(=O)O)O[C@@H]2COC(C)=O)O[C@H](CO)[C@@H](O)[C@@H]1O] |
| 6 | 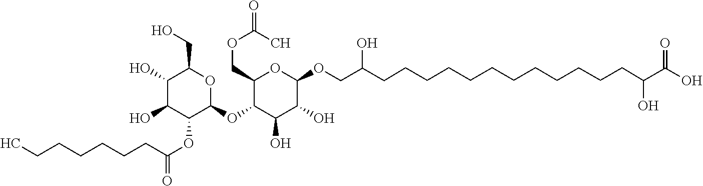 [SMILES: CCCCCCCC(=O)O[C@H]1[C@H](O[C@H]2[C@H](O)[C@@H](O)[C@H](OCC(O)CCCCCCCCCCCCC(O)C(=O)O)O[C@@H]2COC(C)=O)O[C@H](CO)[C@@H](O)[C@@H]1O] |
| | | Molecular | |
|---|---|---|---|
| Compound | Formula | weight | CAS |
| 1 | C₃₆H₆₄O₁₈ | 784.89 | 867578-35-6 |
| 2 | C₃₈H₆₈O₁₈ | 812.94 | 1427087-16-8 |
| 3 | C₃₆H₆₄O₁₇ | 768.89 | 1427028-86-1 |
| 4 | C₃₈H₆₈O₁₇ | 796.95 | |
| 5 | C₃₆H₆₄O₁₇ | 768.89 | |
| 6 | C₃₈H₆₈O₁₇ | 796.95 | 1427039-33-5 |

or a salt of said glycolipid.

7. The method of any of claims 3 to 6 wherein said salts represent alkali salts, alkaline earth salts, ammonium salts, alkanol ammonium salts, glucammonium salts or mixtures thereof.

8. The method of claim 1, wherein said at least one glycolipid is added in an amount of from about 1 to about 1,000 ppm—calculated on the composition.

9. The method of claim 1, wherein said at least one glycolipid is added an amount of from about 10 to about 200 ppm—calculated on the composition.

10. The method of claim 1, wherein secondary anti-microbial agents are added selected from the group consisting of benzoic acid and para-hydroxybenzoic acid, their esters and salts, benzyl benzoate, propionic acid and its salts, salicylic acid and its salts, 2,4-hexadienoic acid (sorbic acid) and its salts, levulinic acid and its salts, anisic acid and its salts, perillic acid and its salts, cinnamic acid and its salts, formaldehyde and paraformaldehyde, 4-hydroxy benzaldehyde, ortho-, meta-, and para-anisic aldehyde, cinnamic aldehyde, cinnamic alcohol, 2-hydroxybiphenyl ether and its salts, 2-zinc-sulfidopyridine N-oxide, inorganic sulfites and bisulfites, sodium iodate, chlorobutanolum, 4-ethylmercury-(II)5-amino-1,3-bis(2-hydroxybenzoic acid), its salts and esters, dehydracetic acid, formic acid, 1,6-bis(4-amidino-2-bromophenoxy)-n-hexane and its salts, the sodium salt of ethylmercury-(II)-thiosalicylic acid, phenylmercury and its salts, 10-undecylenic acid and its salts, 5-amino-1,3-bis(2-ethylhexyl)-5-methyl-hexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)-urea, 4-chloro-m-cresol, 2,4,4′-trichloro-2′-hydroxy-diphenyl ether, 4-chloro-3,5-dimethyl phenol, 1,1′-methylene-bis(3-(1-hydroxymethyl-2,4-dioximidazolidin-5-yl)urea), poly-(hexamethylenediguanide) hydrochloride, (Benzyloxymethoxy)-methanol hexamethylenetetramine, 1-(3-chloroallyl)-3,5,7-triaza-1-azonia-adamantane chloride, 1-(4-chlorophenoxy)-1-(1H-imidazol-1-yl)-3,3-dimethyl-2-butanone, 1,3-bis-(hydroxymethyl)-5,5-dimethyl-2,4-imidazolidinedione, 1,2-dibromo-2,4-dicyanobutane, 2,2′-methylene-bis(6-bromo-4-chlorophenol), bromochlorophene, mixture of 5-chloro-2-methyl-3(2H)-isothiazolinone, 2-methyl-3(2H)-isothiazolinone and with magnesium chloride and magnesium nitrate, 2-Octyl-2H-isothiazol-3-one, 1,2-benzisothiazol-3(2H)-one, 2-benzyl-4-chlorophenol, 3-(4-Chlorphenoxy)-1,2-propanediol (Chlorphenesin), 2-chloroacetamide, chlorhexidine, chlorhexidine acetate, chlorhexidine gluconate, chlorhexidine hydrochloride, N-alkyl(C₁₂-C₂₂)trimethyl-ammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxy-methylurea, 1,6-bis(4-amidino-phenoxy)-n-hexane and its salts, glutaraldehyde, 5-ethyl-1-aza-3,7-dioxabicyclo(3.3.0)octane, 3-(4-chlorophenoxy)-1,2-propanediol, hyamines, alkyl-(C₈-C₁₈)-dimethyl-benzyl-ammonium chloride, alkyl-(C₈-C₁₈)-dimethyl-benzylammonium bromide, alkyl-(C₈-C₁₈)-dimethylbenzyl-ammonium saccharinate, benzyl hemiformal, 3-iodo-2-propynyl butylcarbamate, sodium hydroxymethyl-aminoacetate or sodium hydroxymethyl-aminoacetate, imidazolidinylurea, diazolidinylurea, sodium hydroxymethylglycinate, DMDM hydantoin, Tropolone, (Ethylendioxy)dimethanol, 2-Brom-2-(brommethyl)pentandinitril, N-(3-Aminopropyl)-N-dodecylpropan-1,3-diamin, α,α′,α″38-trimethyl-1,3,5-triazine-1,3,5(2H,4H,6H)-triethanol, pyridine-2-thiol-1-oxide, sodium salt, Tetrahydro-1,3,4,6-tetrakis(hydroxymethyl)imidazo[4,5-d]imidazol-2,5(1H,3H)-dion, 1,3-bis(hydroxymethyl)-1-(1,3,4-tris(hydroxy-methyl)-2,5-dioxoimidazolidin-4-yl)urea (Diazolidinyl Urea), 1,3-Bis(hydroxy-methyl)-5,5-dimethylimidazolidine-2,4-dione, 3-Acetyl-2-hydroxy-6-methyl-4H-pyran-4-one, cetyl pyridium chloride, caprylhydroxamic acid, sorbohydroxamic acid and their mixtures; 1,3-propanediol, methyl propanediol, 1,2-pentanediol, 1,2-hexanediol, 1,2-octanediol, 1,2-decanediol, 1,5-pentanediol, 1,6-hexanediol, 1,8-octanediol, 1,2-decanediol, ethylhexylglycerin, hexoxy-propan-1,2-diol, heptoxy-propan-1,2-diol, octoxy-propan-1,2-diol, 3-phenoxy-propan-1,2-diol, 3-benzyloxy-propan-1,2-diol, 3-phenylethyloxypropan-1,2-diol, 3-phenylpropyloxy-propan-1,2-diol, 3-methylbenzyloxy-propan-1,2-diol, sorbitan caprylate, triclosan, climbazole, Octopirox (1-hydroxy-4-methyl-6-(2,4,4-trimethylpentyl)-2(1H)-pyridone, 2-aminoethanol), chitosan, farnesol, 2-butyloctanoic acid, 2-Benzylheptan-1-ol, glycerol monolaurate, bis(2-pyridylthio)zinc 1,1′-dioxide, N,N′-(decane-1,10-diyldipyridin-1-yl-4-ylidene)-dioctan-1-amine dihydrochloride (octenidine dihydrochloride), thymol, eugenol, benzyl alcohol, 2-phenyethyl alcohol, 3-phenyl propanol, 2-phenoxyethanol, 1-phenoxy-propan-2-ol, 3-phenoxypropanol, benzyloxymethanol, 4-hydroxyacetophenone and mixtures thereof.

11. The method of claim 1, wherein solubility affecting agents are selected from the group consisting of
saponines obtainable from the plant genera
Quillaja,
Yucca,
Dioscorea,
Paris,
Hosta,
Agave,
Asparagus,
Allium,
Sapindus,
Aesculus,
Hedera,
Blighia,
Dipsacus,
Aralia and/or
Anemone
or mixtures thereof,
emulsifiers E400 to E499 or E999 approved for food preparations in the EU, or mixtures thereof
synthetic food grade emulsifiers selected from the group consisting of lecithin; polyglycerol esters (PGE), polysorbates, stearoyl lactylates, propylene glycol esters (PGMS), sucrose esters; polyglycerol Polyricinoleate (PGPR); ammonium phosphatide (AMP); mono and diglycerides of C₆-C₂₂ saturated or unsaturated fatty acids and mixtures thereof.

12. The method of claim 1, wherein the pH value of the composition is adjusted by inorganic or organic acids to about 1 to about 4.

13. The use of glycolipids as antimicrobial agents against at least one of the following microorganisms
Asaia siamensis
Bacillus licheniformis
Bacillus subtilis
Byssochlamys fulva
Byssochlamys nivea
Candida parapsilosis
Clostridium perfringens
Clostridium sporogenes
Corynebacterium xerosis
Enterococcus faecalis
Lactobacillus paracasei ssp. paracasei
Leuconostoc lactis
Leuconostoc mesenteroides
Malassezia furfur
Neosartorya fischeri
Penicillium paneum
Saccharomyces cerevisiae
Yarrowia lipolytica
Zygosaccharomyces bailii
for preserving compositions at a pH of about 1 to about 4,
wherein said glycolipid represents an ustilagic acid or an ester of ustilagic acid esterified with a linear C₁-C₄ aliphatic alcohol.

14. The use according to claim 13, wherein said at least one ustilagic acid or its ester is added in a working amount of from about 1 to about 1,000 ppm—calculated on the composition.

15. The use according to claim 13, wherein said composition is a cosmetic, detergent or food preparation.
